(19) United States
(12) Patent Application Publication
(10) Pub. No.: US 2026/0207671 A1
(43) Pub. Date: Jul. 23, 2026

(54) STEM CELL FORMULATION COMPOSITION, MASS PRODUCTION METHOD AND CLINICAL APPLICATION THEREOF

(71) Applicant: BEIJING ZEPHYRM BIOTECHNOLOGY CO., LTD., Beijing (CN)

(72) Inventors: Yu ZHANG, Beijing (CN); Yidi GAN, Beijing (CN); Yanchao LUO, Beijing (CN); Yibo ZHANG, Beijing (CN); Yan ZHANG, Beijing (CN); Shuai ZHANG, Beijing (CN); Yangmiao YE, Beijing (CN); Shuyan WANG, Beijing (CN); Ranran XU, Beijing (CN); Yichao WANG, Beijing (CN)

(21) Appl. No.: 19/142,124

(22) PCT Filed: Nov. 22, 2023
(86) PCT No.: PCT/CN2023/133281

§ 371 Date: Jun. 20, 2025

(30) Foreign Application Priority Data
Dec. 21, 2022 (CN) 202211648405.7

(51) Int. Cl. A61K 35/28; A61K 47/02; A61K 47/12; A61K 47/18; A61K 47/20; A61K 47/22; A61K 47/26; A61K 47/36; A61K 47/42; C12N 5/0775

(52) U.S. Cl. CPC A61K 35/28; A61K 47/02; A61K 47/12; A61K 47/183; A61K 47/20; A61K 47/22; A61K 47/26; A61K 47/36; A61K 47/42; C12N 5/0662; C12N 2506/02

(57) ABSTRACT

The present invention relates to a stem cell formulation composition, a mass production method thereof, and clinical applications thereof. In the present invention, the uniform and stabilized human embryonic stem cells are used as seed cells to further explore the resuspension, washing, formulation, cryopreservation, and clinical application, along with production process flow and process parameters, in order to solve the problems of large-scale production of MSC formulations and off-the-shelf MSC formulations. The present invention provides an off-the-shelf MSC formulation composition, and also a preparation process for producing the composition, which is specifically a full set of process flow comprising resuspension, washing, formulation preparation, filling, programmed freezing, cryopreservation, and blending for clinical application after large-scale expansion and harvesting of MSCs. The various formulation combinations and process flow parameters developed by the present invention for large-scale production of clinical off-the-shelf MSC formulations are comprehensive in content and have excellent actual production effects and yields.

### TECHNICAL FIELD

The present invention relates to the field of biomedicine, and specifically to a stem cell formulation composition, mass production method and clinical application thereof.

### BACKGROUND ART

Mesenchymal stromal/stem cell (MSC) formulations are generally limited by the problem of cell formulation stability and cannot be used immediately, so that they often need to be cultured and expanded in vitro and then injected clinically within a few hours, that is, they cannot be stored for a long time, which limits the shelf life of MSC formulations. Another problem with MSC formulations is that their cell activity is insufficient after freezing, which limits their biological function.

At present, the batch size and scale of MSC formulations are limited. On the one hand, many MSCs are difficult to produce on a large scale because they are derived from adults, which limits the number of passages and the quantity of cells that can be expanded. On the other hand, the stability of cells in the formulation solution is insufficient to support the time required for bulk filling, leading to batch size limitations. The components and ratios thereof in reagents used for cell resuspension and washing in the present production process of MSC formulations are not sufficiently studied. Commonly, the cells are resuspended in their own complete culture medium and washed with PBS and DPBS, which leads to a significant decrease of cell viability in short-term, so that the available time reserved for subsequent operations is seriously insufficient, and large-scale production cannot be achieved. Additionally, the components of reagents for resuspension and washing are quite different from the components of reagents for subsequent cryopreservation, which further increases the risk of impurity residues in the process.

MSC formulations also have the problem of excipient safety: excipients containing human components such as human serum albumin, human platelet extracts, etc., are generally used as cell nutrition components in the industry, but they are prone to exogenous factor contamination and bring safety risks, which is not conducive to clinical application.

In addition, the existing manufacture process of MSC formulations can cause cell damage: it is inevitable to add human, animal or recombinant cytokines, growth factors and other components during cell culture, which need to be fully removed by multiple washing steps before formulations are made. However, it is difficult to maintain the stability of cells in washing solutions, and cell damage occurs during the washing process, resulting in a decrease in cell activity and viability.

Moreover, for MSC formulations, the cell density for clinical infusion should not be too high. There are usually two options for off-the-shelf cell formulation. One option is to use cryopreservation formulation that can be resuscitated and then directly infused, but usually a large infusion volume is required, which inevitably leads to a large amount of dimethyl sulfoxide (DMSO) in a single infusion that is easy to cause safety risks. The other option is to prepare formulation with higher cell density and smaller filling volume, after the cryopreservation formulation are thawed, they are used after being mixed at a fixed density, but the cell damage caused by the sudden decrease in osmotic pressure after mixing is generally not considered.

Existing research in the industry has not fully considered clinical mixing solutions suitable for the application of MSC formulation, and thus the demand for immediate use of cellular product is not met.

In this field, commonly used stem cell drug packaging materials, such as PVC soft bags, non-PVC composite film soft bags, etc., on the market are of larger size, and are not suitable for cell formulations with smaller filling volumes.

The problem of immediate application of MSC formulations has been mentioned in previous literature, but it is limited to the one-sided optimization of cell cryopreservation formulations, which is far from fully solving the problem of clinical off-the-shelf of MSC formulation. As for the problem of achieving large-scale production of MSC formulations, it has been never mentioned in the literature.

CNT12538456A discloses a pluripotent stem cell, pharmaceutical composition, preparation method thereof and use thereof, which describes a method for producing a MSC population, a MSC population produced by the method, and a culture supernatant thereof, as well as a pharmaceutical composition containing such cells or their culture supernatant. CN112538456A systematically describes how to use existing technical conditions and different types of culture media under laboratory conditions to produce MSCs based on stem cells in vitro that meet the cytokine secretion amount, and clearly indicates the various types of components of culture media required for in vitro differentiation and culture of MSCs and their application fields, which has important guiding significance for in vitro differentiation and culture of MSCs in laboratory-scale. However, CN112538456A only describes the laboratory-scale differentiation and cultivation of MSCs with sufficient secretion of relevant cytokines, and does not explore the entire process of clinical application of MSCs, and does not fully demonstrate and describe how to mass-produce stem cells that meet pharmaceutical standards and related formulations, such as whether the components of the culture media used meet clinical standards and how the obtained cells can be safely and effectively used in clinical practice.

CN110973119A discloses a method for cryopreservation of umbilical cord MSC cryopreservation formulation, which provides a cryopreservation freezing program method for umbilical cord MSC cryopreservation formulation. CN110973119A has summarized a programmed freezing method suitable for umbilical cord MSCs through relevant experimental verification, which can make the cell survival rate and the cell quality after recovery almost close to the cell quality before cryopreservation, and has certain guiding significance for the cryopreservation of MSCs. However, the freezing program of CN110973119A is only applicable to the programmed freezing of umbilical cord MSCs, which is not universal, and does not take into account the MSCs from different sources and does not clearly explain the influence of the components of the cryopreservation formulation used on the cell parameters after programmed freezing. The single demonstration of the programmed freezing step has limited reference significance for the large-scale production of stem cell drugs.

CN112741081A relates to the field of stem cell technology, and discloses a programmed freezing method for human umbilical cord MSCs with excellent cryopreservation cooling effect. It specifically provides a cryopreservation solution to explore the programmed freezing method of human umbilical cord MSCs. Preferably, the cryopreservation solution comprises at least 20% to 50% human albumin, 5% DMSO, 40% to 60% compound electrolyte solution, 1% to 10% sodium chloride solution, and 1% to 10% glucose solution by volume percentage. CN112741081A provides a clear method for programmed freezing of MSCs using a cell cryopreservation solution with clear components. At the same time, the cryopreservation solution is serum-free cryopreservation solution with clearly indicated cryopreservation solution components and explicitly demonstrated functions, and without animal-derived components. After the cells were frozen for three days by the programmed freezing step, the cells were revived with a significant viability advantage (97%). However, although the cryopreservation solution of CN112741081A avoids animal-derived components, it still contains human albumin, a human-derived component, and still has risks in clinical application. In the operation process of the examples, trypsin was frequently used to remove the cells from the wall, which directly introduced animal-derived components. Although the umbilical cord MSCs have a high viability, they still have a high safety risk for clinical application.

CN102487939A relates to the field of cell biology and discloses a MSC storage solution, in which a specifically disclosed MSC storage solution comprises glucose, sodium chloride, albumin, propylene glycol, cholesterol, sodium bicarbonate and other components, and may also comprise heparin component. It is achieved by the MSC storage solution provided by CN102487939A that MSCs can be stored at −20 to 4° C. for 14 to 20 days with a viability of more than 85%, which facilitates the transportation and temporary storage of the cells; and the storage solution components are all from medical injectable reagents that are safe for the human body, and can be directly injected after thawing, thereby facilitating the long-distance supply and clinical application of MSC formulation. However, although CN102487939A uses pharmaceutical-grade reagents that are safe for the human body, the sources thereof should be further indicated to avoid human and animal-derived components as much as possible. Secondly, even the source of single cell storage solution component is explicit, it is still not enough to ensure that the target product meets the stem cell standard of clinical medicinal grade. Strict standard requirements should be set for related formulation in all steps such as stem cell culture, harvesting, washing, and resuspension. In addition, the preparation of the storage solution is relatively cumbersome, and batch differences are prone to occur, which affects the freezing efficiency and clinical use of the cells. In addition, the storage condition of −20 to 4° C. only supports short-term storage, and still cannot meet the requirements for long-term storage and off-the-shelf of large quantities of MSCs.

CN112655702A discloses a solution for umbilical cord MSCs, an umbilical cord MSC formulation, a formulation preparation method and use thereof, which belong to the field of application of umbilical cord MSCs, and in particular to a solution for umbilical cord MSCs and use thereof, an umbilical cord MSC formulation for treating ulcerative colitis and a preparation method and use thereof. The invention provides a solution for umbilical cord MSCs, wherein the solution for umbilical cord MSCs comprises clinical-grade DMSO, human albumin, low-molecular-weight dextran, trehalose, melatonin and a compound electrolyte solution. In the solution for umbilical cord MSCs, the volume percentage of clinical-grade DMSO is 5% to 10%, the mass concentration of human albumin is 1 to 6 g/mL, the mass concentration of low-molecular-weight dextran is 1 g/L, the mass concentration of trehalose is 1 to 5 g/L, and the mass concentration of melatonin is 0.05 to 0.5 g/L. The solution mentioned in CN112655702A can effectively stabilize the number of frozen umbilical cord MSCs, and can also effectively improve the cell viability of frozen umbilical cord MSCs. At the same time, it can maintain the immunosuppressive function of frozen umbilical cord MSCs, which is convenient for long-distance transportation and use of solutions containing umbilical cord MSCs, and can effectively promote the industrial production and use of umbilical cord MSCs. However, the components described in CN112655702A comprise human-derived human albumin, which increases the risk of clinical application of the stem cell formulation; at the same time, CN112655702A does not mention the shelf life of the prepared stem cell formulation and the effects on key parameters such as cell viability during the shelf life, and only mentions its application effect in a mouse model with relatively broad and general contents.

Haack-Sorensen M, Ekblond A, and Kastrup J., Cryopreservation and Revival of Human Mesenchymal Stromal Cells. Methods Mol Biol. 2016; 1416:357-74, describe in detail the specific operation process and related reagents used for cryopreservation of MSCs. The description of the steps of cell culture, harvesting, resuspension, washing and programmed freezing in this document has certain guiding significance for the operation of large-scale production of stem cell products; including the selection of reagents, instrument parameters, formulation packaging materials, etc. used in the operation process, which has certain directive significance. However, the cell culture and cryopreservation formulations described in this document contain a variety of human and animal components, such as FBS, human serum lysate, human serum albumin, etc., and there is a lack of research on the effects of cryopreservation formulation on important parameters such as cell viability.

Matsumura K, Hayashi F, Nagashima T, and Hyon SH., Long-term cryopreservation of human mesenchymal stem cells using carboxylated poly-l-lysine without the addition of proteins or dimethyl sulfoxide. J Biomater Sci Polym Ed. 2013; 24(12):1484-97, investigate the cell viability and differentiation ability of human bone marrow MSCs after cryopreservation in a new DMSO- and protein-free cell cryopreservation medium. The main component of the cryoprotectant mentioned in the document is carboxylated poly-l-lysine (COOH-PLL), which is produced by the reaction between c poly-l-lysine and succinic anhydride. Unlike the traditional cryopreservation mechanism, this component is adsorbed on the stem cell membrane and does not enter the stem cell. After the bone marrow MSCs used have been stored in 7.5% (w/w) COOH-PLL cryopreservation solution at −80° C. for 24 months, the cell viability is still greater than 90% and the cell proliferation and differentiation ability are well maintained. It is a promising alternative component of cell cryopreservation solution. However, although the main component of the new cryopreservation solution described in the document can reduce the impact of DMSO and protein components on cell activity and the risk of clinical application, the component itself still lacks sufficient verification for clinical use.

Zhang Fengli, Study on cryopreservation manufacture process of human adipose-derived MSCs [D]. Shanghai Jiaotong University, 2017, uses a comprehensive evaluation system to study the preservation solution, preservation time and cell concentration of human adipose-derived MSCs (ADSC) cryogenically stored at 2 to 8° C., and obtains a cryogenic preparation scheme that can ensure the viability and function of ADSC before clinical transplantation and minimize adverse reactions. In this paper, a comprehensive evaluation system is established by detecting cell viability and aggregation rate, cell apoptosis, cell adhesion, clone formation, cell proliferation, osteogenic and adipogenic differentiation, cell immune phenotype, cell cycle, IDO1 expression and Kyn concentration, and the key factors in the preparation process, namely, preservation solution, preservation time and cell density, are comprehensively evaluated to formulate an optimal stem cell cryopreservation formulation preparation scheme, thereby providing a feasible scheme for the application of stem cells in clinical treatment, wherein 5% human serum albumin+compound electrolyte injection is the best preservation solution, the best preservation time is within 24 h, 5×10⁶ cells/ml is the most suitable cell preservation concentration, the late cell apoptosis rate is low, the proliferation is fast, and the osteogenic and adipogenic differentiation ability is good. However, the cell formulation described in this paper is a fresh cell formulation, not an off-the-shelf cell formulation, and has a short shelf life.

Xu Wenjing, Research on mechanisms affecting activity of umbilical cord MSCs and protective solutions thereof [D], Shanxi Medical University, 2014, studies the effects of the five solutions currently commonly used in clinical practice, namely 0.9% sodium chloride injection, 5% glucose injection, compound electrolyte injection (Plasmalyte A), 1% human albumin solution, and 5% human albumin solution, on UC-MSC. In this paper, the effects of different protective solutions on cell activity, cell apoptosis/death rate, cell adhesion ability after cell preservation, cell surface markers, and osteogenic and adipogenic abilities are evaluated. It has certain reference significance for clinical transplantation. In this paper, a self-prepared protective solution formula is also disclosed, and its use effect is verified, wherein 0.1 μg/L insulin, 10⁻⁸ mol/L estrogen and 10⁻⁸ mol/L progesterone, 10⁻⁶ mol/L hydrocortisone, 30 nmol/L sodium selenite, 0.5 mmol/L adenosine, 1 mmol/L N-acetylcysteine, and 0.5 mmol/L L-glutamine are added to 0.9% sodium chloride solution for injection. However, the cell formulation disclosed in this paper is also a fresh cell formulation with limited shelf life. In addition, the proportions of components in the protective solution formula disclosed in this paper have not been fully verified, only the functions of these components are explained before implementation, and the experimental results are not very reliable since there is a lack of relevant data for support. At the same time, the estrogen, progesterone and other ingredients contained in the protective solution greatly increase the risk in clinical use, and make the preparation process cumbersome, and the cell viability after 12 hours is average, which are easy to cause batch differences and increase the difficulty of production operations.

Therefore, large-scale production of off-the-shelf MSC formulation is still needed in this field.

### SUMMARY

In view of the aforementioned problems in the prior art fields, the present invention provides a MSC (M cell) formulation composition that is easy to mass produce, free of human- or animal-derived excipients, stable during cryopreservation, and capable of enabling immediate clinical application and large-scale production of MSC formulations.

In the present invention, the uniform and stabilized human embryonic stem cells are used as seed cells to further explore the resuspension, washing, formulation, cryopreservation, and clinical application, along with production process flow and process parameters, in order to solve the problems of large-scale production of MSC formulations and off-the-shelf MSC formulations.

The present invention aims to solve the problems of the short expiry period of MSC product, small batch production, safety risks caused by the use of human and animal-derived product excipients, unstable cell activity caused by formulation preparation, filling and freezing processes of MSC product in the field, and provides an off-the-shelf formulation composition of MSCs, and also a preparation process for producing the composition, which is specifically a full set of process flow comprising resuspension, washing, formulation preparation, filling, programmed freezing, cryopreservation, and blending for clinical application after large-scale expansion and harvesting of MSCs.

The present invention relates to a stem cell formulation composition, which comprises M cells obtained by directed differentiation and expansion of human embryonic stem cells and an excipient, wherein the excipient comprises one or more of the following:
(a) a permeable protective agent: DMSO;
(b) a non-permeable protective agent: adenosine, low-molecular-weight dextran, lactobionic acid, HEPES, reduced L-glutathione, glucose, sucrose, mannitol, compound amino acids, plant-derived recombinant human serum albumin, etc.;
(c) a crystal solution and a pH regulator: sodium hydroxide, sodium gluconate, sodium acetate, potassium hydroxide, sodium chloride, calcium chloride, magnesium chloride, potassium chloride, potassium bicarbonate, potassium phosphate, calcium chloride, magnesium chloride, etc.

The formulation composition of the present invention may further comprise hydroxyethyl starch, SUL-109 (6-chromanol derivative), malate buffer, acetate buffer, etc.; and their ratio combinations may be endless.

The main active ingredient of the formulation composition of the present invention is the M cells obtained by directed differentiation and expansion of human embryonic stem cells, and the M cell exhibits the characteristics and biological functions of MSCs. After harvesting and washing, the M cells are added with formulation solution components, mixed to form a formulation of about 5×10⁷ cells/ml, filled into cryovials, subjected to programmed freezing to ≤−70° C., and transferred into a vapor-phase liquid nitrogen tank for long-term storage.

The present invention also relates to a batch manufacture process of stem cell formulation, specifically comprising: harvesting centrifugation, resuspension, washing, formulation preparation, filling, programmed freezing and cryopreservation processes after the M cells obtained by directed differentiation and expansion of human embryonic stem cells are harvested, wherein:
(a) resuspension process: comprising a resuspension solution and the process parameters (volume, time and temperature for stable placement, etc.);
(b) washing process: comprising a washing solution and process parameters (number of washing times, replacement volume, centrifugal speed, etc.);
(c) formulation preparation process: comprising cell number, procedure of adding excipient, time and temperature for stable placement, and process monitoring parameters;
(d) programmed freezing process: comprising multiple cooling programs to ensure low-temperature transportation and cryopreservation process.

The process further comprises:
(e) in use process for clinical: blending parameters, stable placement time limit after blending, etc.

The specific process flow of the present invention is as follows:
1. Harvesting M cells: M cells cultured in a cell factory are digested with a digestion solution (TrypLE), then the digestion is terminated with a buffer solution, the M cells are collected in a centrifuge bottle, centrifuged, and the supernatant is discarded;
2. Resuspension and washing:
   The M cells collected by centrifugation are added with an appropriate amount of solution and resuspended;
   The obtained cell suspension is washed with an appropriate volume of an appropriate washing solution for a number of cycles in a horizontal centrifuge or a countercurrent gradient centrifugation system to remove undesired process-related impurities in the culture system, such as BSA, TrypLE, etc.;
   Determination of total number of M cells: The viable cell density is determined by the trypan blue method or the fluorescence method, and the total number of M cells is calculated according to the volume of the resuspension;
3. Formulation preparation: After the washing solution is removed from the M cells, a formulation solution is used to prepare a formulation containing 5×10⁷ cells/ml according to the total number of cells measured, and this step needs to be completed at a temperature of 2 to 8° C.
4. Filling: The formulation is filled into sterile cyclic olefin copolymer (COC) vials at 1 ml, 2 ml or 5 ml, then the vials are stoppered and capped, and this step needs to be completed at a temperature of 2 to 8° C.
5. Programmed freezing: The vials filled with the cell formulation are transferred into a programmed freezing instrument, and cooled to ≤−70° C. according to a preset cooling program.
6. Cryopreservation:
   The cell formulation after the programmed freezing is taken out in batch, and quickly transferred to a liquid nitrogen transport vehicle to keep the temperature ≤−70° C.;
   The cell formulation vials are packed in the liquid nitrogen transport vehicle;
   The finished products as packed are placed in a basket and transferred to a vapor-phase liquid nitrogen storage tank.

The solutions involved in the process of the present invention also comprise solutions of different ratios as termination, resuspension and washing solutions.

The horizontal centrifuge involved in the process of the present invention can have a rotation speed of 300×g to 1000×g.

The packaging container involved in the process of the present invention can also be a cryovial, COC (cyclic olefin copolymer), or COP (cyclic olefin polymer) vial.

The cooling procedure involved in the process of the present invention can also follow the following list:

| Procedure step | Parameter | Parameter range |
|---|---|---|
| Step1 | Wait at | 2 to 8° C. |
| Step2 | Wait at Chamber temperature | 2° C. to 8° C. |
| | until sample temperature | 2° C. to 8° C. |
| Step3 | Ramp | 1° C./min |
| | until Chamber | −11° C. to −17° C. |
| Step4 | Ramp | 20° C. to 30° C./min |
| | until Chamber | −45° C. to −65° C. |
| Step5 | Ramp | 8° C. to 16° C./min |
| | until Chamber | −20° C. to −25° C. |
| Step6 | Ramp | 0.8° C. to 1.2° C./min |
| | until Chamber | −40° C. to −50° C. |
| Step7 | Ramp | 8° C. to 12° C./min |
| | until Chamber | −80° C. to −90° C. |

The present invention also relates to clinical treatment for multiple indications, including two modes of administration: local injection and intravenous infusion. The indications include, but are not limited to: osteoarthropathy (e.g., meniscus injury, osteoarthritis, or bone injury), reproductive system disease (e.g., ovarian aging, ovarian insufficiency, endometrial injury, uterine trauma, intrauterine adhesion, or thin uterus), heart disease (e.g., myocardial infarction), lung disease (e.g., idiopathic pulmonary fibrosis, acute respiratory distress syndrome, pneumoconiosis, or pneumonia), skin disease (e.g., psoriasis, skin injury, bedsore, pressure sore, or burn), eye disease (e.g., corneal injury), nervous system disease (e.g., spinal cord injury, cerebral palsy, stroke, Alzheimer's disease, or neuropathic pain), digestive system disease (e.g., inflammatory bowel disease, colitis, Crohn's disease, or irritable bowel syndrome), kidney disease (e.g., anti-glomerular basement membrane disease, diabetic nephropathy, lupus nephritis, or acute nephritis), liver disease (e.g., liver injury, liver fibrosis, hepatitis, cirrhosis, or liver failure), autoimmune disease (e.g., scleroderma, lupus erythematosus, or multiple sclerosis), transplant rejection (e.g., graft-versus-host disease), metabolic disease (e.g., diabetes), etc.

Therefore, in one aspect, the present invention provides an off-the-shelf stem cell formulation composition, which comprises MSCs (i.e., M cells) obtained by directed differentiation and expansion of human embryonic stem cells and an excipient, wherein the off-the-shelf stem cell formulation composition is free of human-derived and animal-derived excipients, wherein optionally, the excipient comprises one or more of the following: an permeable protective agent, an non-permeable protective agent, a crystal solution and a pH regulator, wherein optionally, the permeable protective agent is selected from DMSO, the non-permeable protective agent is one or more selected from the group consisting of adenosine, low-molecular-weight dextran, lactobionic acid, HEPES, reduced L-glutathione, glucose, sucrose, mannitol, compound amino acids, and plant-derived recombinant human serum albumin, and the crystal solution and pH regulator are one or more selected from the group consisting of sodium hydroxide, sodium gluconate, sodium acetate, potassium hydroxide, sodium chloride, calcium chloride, magnesium chloride, potassium chloride, potassium bicarbonate, potassium phosphate, calcium chloride, and magnesium chloride, wherein optionally, the stem cell formulation composition further comprises one or more selected from the group consisting of hydroxyethyl starch, SUL-109 (6-chromanol derivative), malate buffer, and acetate buffer, wherein optionally, the viable cell density of the formulation composition is about 5×10⁷ cells/ml, wherein optionally, the cell viability of the formulation composition is >90%, preferably >95%, more preferably >96%, and more preferably >97%, wherein optionally, the formulation composition is substantially free of human embryonic stem cell residues, substantially free of bovine serum albumin residues and substantially free of non-target differentiated cells, wherein optionally, the formulation composition is non-tumorigenic, wherein optionally, the formulation composition can be stably stored for at least 24 months, wherein optionally, the formulation composition can be stably placed at −80±10° C. for at least 8 weeks.

In one embodiment, the excipient comprises potassium chloride, sodium chloride, magnesium chloride, sodium lactate, and sodium gluconate, wherein optionally, the excipient further comprises glutathione, dextran-40, adenosine, sucrose, mannitol, dextrose, and DMSO.

On the other hand, the present invention provides a method for mass production of off-the-shelf stem cell formulation, which comprises:
a) harvesting MSCs, i.e., M cells, obtained by directed differentiation and expansion of human embryonic stem cells,
b) resuspending and washing the cells obtained in step a),
c) formulating the washed cells into a stem cell formulation at 2 to 8° C., wherein preferably, the density of the stem cell formulation is about 5×10⁷ cells/ml of formulation,
d) filling the formulated stem cell formulation into a stem cell drug packaging material suitable for a smaller volume of cell formulation at 2 to 8° C., wherein optionally, the packaging material is selected from the group consisting of cryopreservation tube, cyclic olefin copolymer (COC) vial, cyclic olefin polymer (COP) vial,
e) cool the filled stem cell formulations to ≤−70° C. using a cooling program,
f) transporting and/or cryopreserving the obtained off-the-shelf stem cell formulation, wherein the off-the-shelf stem cell formulation is free of human-derived and animal-derived excipients.

In one embodiment, step a) comprises washing the cultured cells with sodium lactate Ringer's solution, digesting with a digestion solution, and then stopping the digestion by adding a stop solution, wherein further optionally, the digestion solution is CTS TrypLE, and the stop solution comprises glutathione, dextran-40, adenosine, sucrose, mannitol, glucose, HEPES, potassium chloride, sodium chloride, calcium chloride, magnesium chloride, potassium dihydrogen phosphate, sodium bicarbonate, sodium lactate, wherein optionally, the stop solution comprises 0.1 to 1 mM glutathione, 0.1% to 1% dextran-40, 0.1 to 0.4 mM adenosine, 1 to 4 mM sucrose, 1 to 4 mM mannitol, 0.1 to 1 mM glucose, 1 to 10 mM HEPES, 5 to 20 mM potassium chloride, 80 to 130 mM sodium chloride, 1 to 4 mM calcium chloride, 1 to 5 mM magnesium chloride, 1 to 5 mM potassium dihydrogen phosphate, 1 to 5 mM sodium bicarbonate, 15 to 30 mM sodium lactate, wherein optionally, the stop solution comprises 0.3 mM glutathione, 0.6% dextran-40, 0.2 mM adenosine, 2 mM sucrose, 2 mM mannitol, 0.5 mM glucose, 5 mM HEPES, 10.12 mM potassium chloride, 111.31 mM sodium chloride, 1.23 mM calcium chloride, 1 mM magnesium chloride, 2 mM potassium dihydrogen phosphate, 1 mM sodium bicarbonate, 24.9 mM sodium lactate.

In another embodiment, wherein the resuspension in step b) comprises adding the M cells obtained in step a) to the resuspension solution for resuspension to obtain a cell suspension, and/or
wherein the washing in step b) comprises washing with a horizontal centrifuge or washing with a countercurrent gradient centrifuge, wherein optionally, the horizontal centrifuge has a rotation speed of 300×g to 1000×g.

In another embodiment, the resuspension solution and/or the washing solution comprises sodium chloride, wherein optionally, the resuspension solution and/or the washing solution comprises glutathione, dextran-40, adenosine, sucrose, mannitol, glucose, HEPES, potassium chloride, sodium chloride, calcium chloride, magnesium chloride, potassium dihydrogen phosphate, sodium bicarbonate, sodium lactate, dextrose, wherein optionally, the resuspension solution and/or the washing solution comprises 1 to 40 mM HEPES, 1 to 30 mM potassium chloride, 80 to 130 mM sodium chloride, 1 to 6 mM calcium chloride, 1 to 5 mM magnesium chloride, 1 to 10 mM potassium dihydrogen phosphate, 1 to 8 mM sodium bicarbonate, 15 to 30 mM sodium lactate, 0.1 to 1.5 mM glutathione, 0.01% to 0.2% dextran-40, 0.1 to 1 mM adenosine, 1 to 10 mM sucrose, 1 to 10 mM mannitol, 0.1 to 4 mM dextrose, wherein optionally, the resuspension solution and/or the washing solution comprises 5 to 30 mM HEPES, 4 to 30 mM potassium chloride, 80 to 120 mM sodium chloride, 1.8 to 3 mM calcium chloride, 1 to 4.4 mM magnesium chloride, 2 to 8 mM potassium dihydrogen phosphate, 1 to 4 mM sodium bicarbonate, 22 to 27 mM sodium lactate, 0.3 to 1.2 mM glutathione, 0.06% to 0.18% dextran-40, 0.2 to 0.8 mM adenosine, 2 to 8 mM sucrose, 2 to 8 mM mannitol, 0.5 to 2 mM dextrose.

In another embodiment, the washed cells are formulated into a stem cell formulation using a formulation solution, wherein the formulation solution comprises glutathione, dextran-40, adenosine, sucrose, mannitol, dextrose, HEPES, potassium chloride, sodium chloride, calcium chloride, magnesium chloride, potassium dihydrogen phosphate, sodium bicarbonate, DMSO, wherein optionally, the formulation solution comprises 20 to 30 mM HEPES, 30 to 35 mM potassium chloride, 90 to 100 mM sodium chloride, 0.01 to 0.1 mM calcium chloride, 1 to 10 mM magnesium chloride, 5 to 15 mM potassium dihydrogen phosphate, 1 to 10 mM sodium bicarbonate, 1 to 6 mM glutathione, 2% to 12% dextran-40, 1 to 4 mM adenosine, 15 to 25 mM sucrose, 15 to 25 mM mannitol, 1 to 10 mM dextrose, 1% to 15% DMSO, wherein optionally, the formulation solution comprises 25 mM HEPES, 32.5 mM potassium chloride, 95 mM sodium chloride, 0.05 mM calcium chloride, 5 mM magnesium chloride, 10 mM potassium dihydrogen phosphate, 5 mM sodium bicarbonate, 3 mM glutathione, 6% dextran-40, 2 mM adenosine, 20 mM sucrose, 20 mM mannitol, 5 mM dextrose, 5% DMSO, wherein optionally, the formulation solution further comprises 1 to 10% compound amino acids, optionally 2% compound amino acids, or optionally 5% compound amino acids.

In another embodiment, the programmed freezing comprises: waiting at 2° C. to 8° C.; cooling to −11° C. to −17° C. at 1° C. to 2° C./min; cooling to −45° C. to −65° C. at 20° C. to 30° C./min; heating to −15° C. to −25° C. at 8° C. to 16° C./min; cooling to −35° C. to −45° C. at 1° C. to 2° C./min; cooling to −80° C. to −90° C. at 8° C. to 12° C./min; maintaining at −80° C. to −90° C. for 10 min to 20 min, wherein optionally, the programmed freezing comprises: waiting at 4° C.; cooling to −13.0° C. at 1° C./min; cooling to −55.0° C. at 25° C./min; heating to −20.0° C. at 12° C./min; cooling to −40.0° C. at 1.0° C./min; cooling to −90.0° C. at 10.0° C./min; maintaining at −90.0° C. for 10 min, wherein further optionally, the programmed freezing comprises: waiting at 2° C. to 8° C.; cooling to −11° C. to −17° C. at 1° C. to 2° C./min; cooling to −45° C. to −65° C. at 20° C. to 30° C./min; heating to −20° C. to −25° C. at 8° C. to 16° C./min; cooling to −40° C. to −50° C. at 0.8° C. to 1.2° C./min; cooling to −80° C. to −90° C. at 8° C. to 12° C./min.

In another aspect, the present invention also provides an off-the-shelf stem cell formulation produced by the method of the present invention.

In another aspect, the present invention also provides a use of the off-the-shelf stem cell formulation composition or the off-the-shelf stem cell formulation of the present invention in the manufacture of a medicament for treating an indication, wherein the indication includes but is not limited to: osteoarthropathy (e.g., meniscus injury, osteoarthritis, or bone injury), reproductive system disease (e.g., ovarian aging, ovarian insufficiency, endometrial injury, uterine trauma, intrauterine adhesion, or thin uterus), heart disease (e.g., myocardial infarction), lung disease (e.g., idiopathic pulmonary fibrosis, acute respiratory distress syndrome, pneumoconiosis, or pneumonia), skin disease (e.g., psoriasis, skin injury, bedsore, pressure sore, or burn), eye disease (e.g., corneal injury), nervous system disease (e.g., spinal cord injury, cerebral palsy, cerebral stroke, Alzheimer's disease, or neuropathic pain), digestive system disease (e.g., inflammatory bowel disease, colitis, Crohn's disease, or irritable bowel syndrome), kidney disease (e.g., anti-glomerular basement membrane disease, diabetic nephropathy, lupus nephritis, or acute nephritis), liver disease (e.g., liver injury, liver fibrosis, hepatitis, cirrhosis, or liver failure), autoimmune disease (e.g., scleroderma, lupus erythematosus, or multiple sclerosis), transplant rejection (e.g., graft-versus-host disease), metabolic disease (e.g., diabetes), wherein optionally, the treatment comprises administering the medicament by local injection or intravenous infusion, and wherein further optionally, the drug is encapsulated in a stem cell drug packaging material suitable for cell formulation with a smaller volume.

The technical effects achieved by the present invention are as follows:
(1) No human-derived or animal-derived components are used in the formulation composition and the entire production process, and there is no safety risk of exogenous factors.
(2) The resuspension-washing solutions, formulation compositions and process conditions obtained by screening can ensure that the cells still maintain a high viability and activity during large-scale production of the formulation.
(3) The resuspension-washing process ensures that there is no residue of culture medium components, thereby effectively reducing the safety risks caused by impurities.
(4) The process enables batch production of thousands of vials.
(5) The long-term storage and clinical off-the-shelf capability is achieved, which meets the treatment window requirement for many acute patients.
(6) Clinical preparation can maintain a high activity for several hours, which ensures a sufficient clinical operation window.
(7) It ensures that M cells have quality indicators such as immune regulation, tissue repair, and tridirectional differentiation ability, providing possibilities for clinical treatment, showing good clinical application potential, and being able to be used for the treatment of human diseases, wherein the diseases may include: osteoarthropathy (e.g., meniscus injury, osteoarthritis, or bone injury), reproductive system disease (e.g., ovarian aging, ovarian insufficiency, endometrial injury, uterine trauma, intrauterine adhesion, or thin uterus), heart disease (e.g., myocardial infarction), lung disease (e.g., idiopathic pulmonary fibrosis, acute respiratory distress syndrome, pneumoconiosis, or pneumonia), skin disease (e.g., psoriasis, skin injury, bedsore, pressure sore, or burn), eye disease (e.g., corneal damage), nervous system disease (e.g., spinal cord injury, cerebral palsy, stroke, Alzheimer's disease, or neuropathic pain), digestive system disease (e.g., inflammatory bowel disease, colitis, Crohn's disease, or irritable bowel syndrome), kidney disease (e.g., anti-glomerular basement membrane disease, diabetic nephropathy, lupus nephritis, or acute nephritis), liver disease (e.g., liver injury, liver fibrosis, hepatitis, cirrhosis, or liver failure), autoimmune disease (e.g., scleroderma, lupus erythematosus, or multiple sclerosis), transplant rejection (e.g., graft-versus-host disease), metabolic disease (e.g., diabetes), etc.

The various formulation combinations and process flow parameters developed by the present invention for large-scale production of MSC formulations for clinical use are comprehensive and unique, with excellent actual production effects and yields, which have not been described in previous patent documents and non-patent documents.

### BRIEF DESCRIPTION OF THE DRAWINGS

FIG. 1 shows a process flow diagram of the present invention.

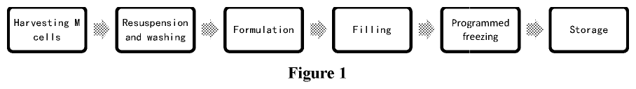

FIG. 2 shows a programmed freezing curve diagram according to an embodiment of the present invention as example.

FIG. 3 shows the results of chromosome karyotype analysis of three consecutive batches of M cell formulations produced according to an embodiment of the present invention as example.

FIG. 4 shows a schematic diagram of the tridirectional differentiation ability of one batch of M cell formulation according to an embodiment of the present invention as example.

FIG. 5 shows the results of immunoregulation on lymphocytes and subsets thereof by the M cell formulation according to an embodiment of the present invention as example.

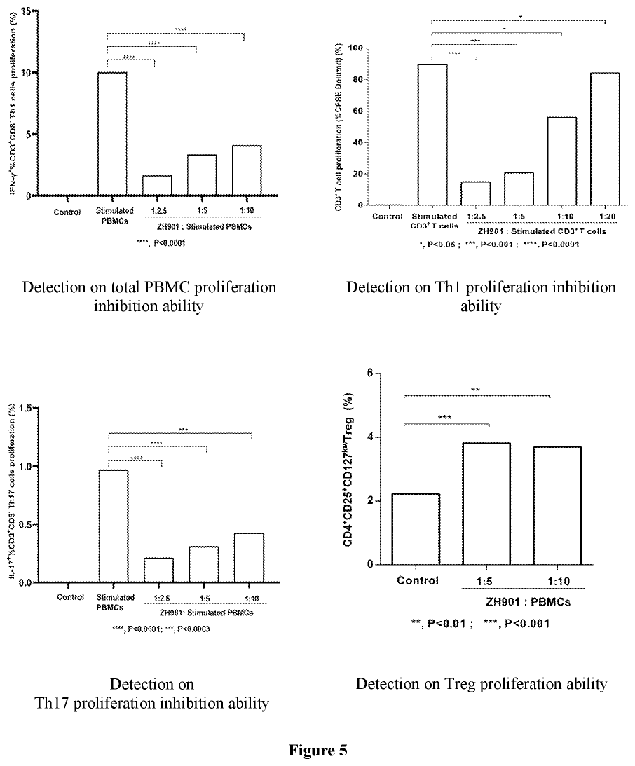

FIG. 6 shows the results of the change trend of cell viability during placement at 2 to 8° C. and the change trend of cell viability during placement at room temperature for the M cell formulation according to an embodiment of the present invention as example after being blended.

### SPECIFIC MODELS FOR CARRYING OUT THE PRESENT INVENTION

The present invention can be carried out through the following exemplary embodiments, but it is known to those skilled in the art that the present invention is not limited to the exemplary embodiments. Those skilled in the art can make corresponding modifications to the exemplary embodiments of the present invention as needed, and the modified embodiments also fall into the scope of the present invention.

Unless otherwise indicated, the reagents used in the present invention are all commercially available reagents. The instruments, equipment, devices, etc. used in the present invention are all conventional instruments, equipment, devices, etc.

The experimental materials involved in the research of the present invention are as follows:

| | | Implementation |
|---|---|---|
| Name | Manufacturer/Source | standard/Excipient grade |
| CTS TrypLE | Thermofisher | For cell therapy |
| CTS DPBS | Thermofisher | For cell therapy |
| Sodium lactate | Shanghai Baite Medical | Marketed drug |
| Ringer's solution | Products Co., Ltd. | |
| Glutathione | Merck | USP/Pharmaceutical grade |
| Dextran-40 | Merck | USP/Pharmaceutical grade |
| Adenosine | Merck | USP/Pharmaceutical grade |
| Sucrose | Merck | USP/Pharmaceutical grade |
| Mannitol | Jiangsu Hansitong | Ch. P/Pharmaceutical grade |
| | Pharmaceutical Co., Ltd. | |
| Dextrose | Merck | USP/Pharmaceutical grade |
| HEPES | Merck | USP/Pharmaceutical grade |
| Trehalose | Jiangsu Hansitong | Ch. P/Pharmaceutical grade |
| | Pharmaceutical Co., Ltd. | |
| Potassium chloride | Merck | USP/Pharmaceutical grade |
| Sodium chloride | Merck | USP/Pharmaceutical grade |
| Calcium chloride | Merck | USP/Pharmaceutical grade |
| Magnesium chloride | Merck | USP/Pharmaceutical grade |
| Potassium dihydrogen phosphate | Merck | USP/Pharmaceutical grade |
| Sodium bicarbonate | Merck | USP/Pharmaceutical grade |
| DMSO | OriGen | USP/Ph. EU/Pharmaceutical |
| | | grade |
| Plant-derived recombinant | Wuhan Heyuan | — |
| human serum albumin OsrHSA | Biotechnology Co., Ltd. | |
| (Recombinant Human Serum | | |
| Albumin from Oryza sativa) | | |
| 20% Human albumin | Shandong Taibang | Ch. P/Marketed drug |
| | Biological Products Co., Ltd. | |
| Compound electrolyte injection | Shanghai Baite Medical | National drug standard |
| | Products Co., Ltd. | WS1- (X-225-2003Z) |
| Compound amino acids | Shijiazhuang Fourth | Marketed drug |
| | Pharmaceutical | |
| 2 ml COP Cyclic olefin resin vial | Gerresheimer | Drug packaging material |
| Brominated butyl rubber stopper | Datwyler | Drug packaging material |
| Aluminum-plastic combination | Datwyler | Drug packaging material |
| cap | | |

### EXAMPLES

### Example 1: Batch Production and Quality Evaluation of Three Batches of M Cell Formulations

Two washing processes were used to prepare three batches of M cell formulations in large quantities, with 50 cell factories produced in each batch, and the batch-to-batch robustness of the present production process and the consistency of product quality were investigated, which were specifically described as follows:

### 1.1 Cell Harvesting

Human embryonic stem cells ZH-hESC (the applicant adopted a method widely used in the industry, which was referred to Gu Q, et al. Accreditation of Biosafe Clinical-Grade Human Embryonic Stem Cells According to Chinese Regulations. Stem Cell Reports. 2017 Jul. 11; 9(1):366-380, to establish a cell bank for production through the development of fertilized eggs obtained by in vitro fertilization for 3 to 7 days to form blastocysts, isolation of inner cell masses therefrom, in vitro expansion to establish a cell line, and identification) was resuscitated in a culture medium (containing Essential 8 ™ (E8) basal medium and Essential 8 ™ supplementary medium), cultured for 5 to 7 days, and passaged once; embryoid bodies were prepared, and cultured for 3 to 7 days, then inoculated into a MSC differentiation medium (containing α-MEM, 5% KOSR, 1% Ultroser G, CTS GlutMAX, 1×NEAA, l0 ng/ml bFGF, 4 ng/ml rhTGF-β, 10 μg/ml VC) according to a specified number (20 embryoid bodies/10 cm² of culture dish), differentiated for about 10 to 20 days, and then continuously passaged at an appropriate density (1×10⁴ cells/cm²) to P5 generation. When the cells reached 80% to 90% confluence as observed under a microscope, the cells were harvested, with 50 cell factories in each batch.

### 1.2 Resuspension-Washing

The culture medium was discarded from the cell factories, 300 mL of sodium lactate Ringer's solution was added to each cell factory for rinsing once and discard, then 200 mL of CTS TrypLE was added to each cell factory for digestion, until the cells were observed to completely detach from the surface of cell factory, then 200 mL of stop solution was added to stop the digestion, the cell suspension was collected into a centrifuge tube, and centrifuged at 800×g for 15 min, and the supernatant was discarded after centrifugation.

1.2.1 Preparation of stop solution and resuspension-washing solution: The stop solution and resuspension-washing solution were prepared according to the following formula and pre-cooled at 2 to 8° C.

| | | | Component of | |
|---|---|---|---|---|
| | Component of | | resuspension- | |
| Component | stop solution | | washing solution | |
| Glutathione | 0.3 | mM | 0.15 | mM |
| Dextran-40 | 0.6% | | 0.03% | |
| Adenosine | 0.2 | mM | 0.01 | mM |
| Sucrose | 2 | mM | 1 | mM |
| Mannitol | 2 | mM | 1 | mM |
| Glucose | 0.5 | mM | 0.25 | mM |
| HEPES | 5 | mM | 2.5 | mM |
| Potassium chloride | 10.12 | mM | 7.07 | mM |
| Sodium chloride | 111.31 | mM | 107 | mM |
| Calcium chloride | 1.23 | mM | 1.30 | mM |
| Magnesium chloride | 1 | mM | 0.5 | mM |
| Potassium dihydrogen phosphate | 2 | mM | 1 | mM |
| Sodium bicarbonate | 1 | mM | 0.5 | mM |
| Sodium lactate | 24.9 | mM | 24.9 | mM |

### 1.2.2 Washing

### 1.2.2.1 Washing Batch 01 and Batch 02 by Horizontal Centrifuge

400 mL of resuspension-washing solution was added to the cell pellet collected from each cell factory to resuspend the cells, then centrifugation was carried out at 800×g for 8 min, the supernatant was discarded after centrifugation, washing was repeated twice, and the total number of cells was measured before the last washing and centrifugation.

### 1.2.2.2 Washing Batch 03 by Countercurrent Gradient Centrifuge

The cell pellet collected from each cell factory was resuspended in an appropriate amount of resuspension-washing solution and washed according to the following parameters:
Resuspension-washing solution volume: 300 mL per cell factory.
Setting of centrifugal force and flow rate for sample loading: at centrifugal force of 2800×g, the sample loading flow rate was 45 mL/min.
Centrifugal force and flow rate for washing: at centrifugal force of 2500×g, 45 mL/min.

Fifty cell factories were processed for each of the three batches, and the total time consumed for the resuspension and washing processes was 1 h42 min, 1 h53 min, and 1 h39 min, respectively, which was well controlled within 2 hours.

### 1.3 Formulation Preparation

### 1.3.1 the Preparation of Formulation Solution

The formulation solution was formulated according to the components and addition amounts in the following table, and pre-cooled at 2 to 8° C.

| Component | Component content | |
|---|---|---|
| Glutathione | 3 | mM |
| Dextran-40 | 6% | |
| Adenosine | 2 | mM |
| Sucrose | 20 | mM |
| Mannitol | 20 | mM |
| Dextrose | 5 | mM |
| HEPES | 25 | mM |
| Potassium chloride | 32.5 | mM |
| Sodium chloride | 95 | mM |
| Calcium chloride | 0.05 | mM |
| Magnesium chloride | 5 | mM |
| Potassium dihydrogen phosphate | 10 | mM |
| Sodium bicarbonate | 5 | mM |

| | |
|---|---|
| DMSO | 5% |

### 1.3.2 the Preparation of Cell Formulation

The washed cells were resuspended in an appropriate amount of the above formulation solution to maintain a high density (not less than 5×10⁷ cells/ml), and mixed well, then a sample was taken to measure the live cell density (A), which was used together with the added formulation solution volume (Va) to determine the supplemented formulation solution volume (Vx) according to the following formula:

$$Vx = \frac{A \times Va}{50000000} - Va$$

The formulation solution was supplemented according to the calculated Vx so that the live cell density in the formulation was about 5×10⁷ cells/ml.

### 1.3.3 Filling

In the filling process, the cell formulation in the storage bottle was always placed in a cooler device at 2 to 8° C., and mixed well by gentle rotation during the period. The cell formulation was filled into cyclic olefin polymer (COP) vials at 1 mL/vial using an automatic dispenser, plugged with rubber stopper, capped using a capping machine for sealing, and labeled. The sealing effect was checked using the three-finger method. The filled formulation must be kept at 2 to 8° C. during the entire filling process.

From formulating completion to filling completion, processing time for the three batches were 2 h34 min, 2 h45 min, and 2 h39 min, respectively, all of which did not exceed 3 hours.

### 1.4 Programmed Freezing

The following cooling program was set for the programmed freezing:
1. Wait at 4.0° C.
2. Wait at Chamber=4.0° C. until sample=5.0° C.
3. Ramp 1° C./min until Chamber=−13.0° C.
4. Ramp 25° C./min until Chamber=−55.0° C.
5. Ramp 12° C./min until Chamber=−20.0° C.
6. Ramp 1.0° C./min until Chamber=−40.0° C.
7. Ramp 10.0° C./min until Chamber=−90.0° C.
8. Hold −90.0° C. for 10.0 minutes
9. End

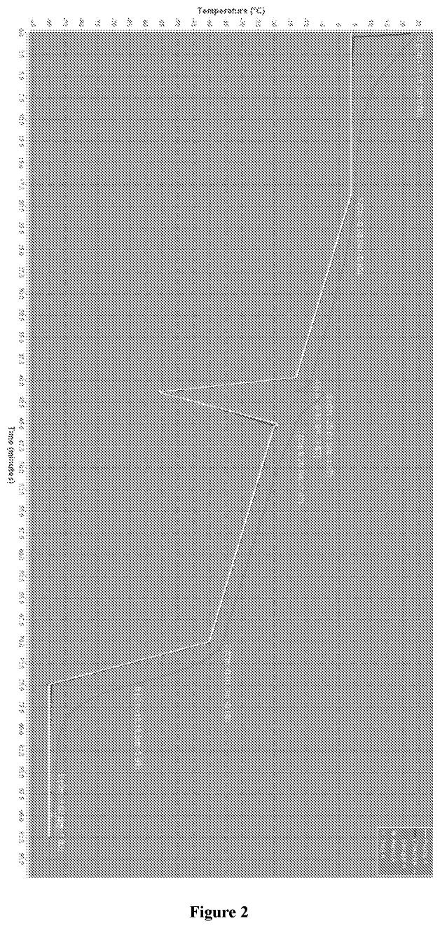

The specific programmed freezing curve was shown in FIG. 2, in which the red line indicated the sample temperature, the white line was the set temperature, and the blue line was the chamber temperature.

### 1.5 Transportation and Cryopreservation

After the programmed freezing was completed, the large batches of cell formulations were quickly placed in a pre-cooled liquid nitrogen transport vehicle, and packed, and that the temperature in the transport vehicle was not higher than −90° C. during the entire operation. Then, it was transferred to a vapor-phase liquid nitrogen tank as soon as possible for long-term storage.

### 1.6 Summary of Production of Three Batches of Formulations

The present production information of the three batches was summarized in the following table:

| Batch No. | Batch 01 | Batch 02 | Batch 03 |
|---|---|---|---|
| Scale | 48 cell factories | 51 cell factories | 49 cell factories |
| Total harvest | 5.02 × 10¹⁰M cells | 5.11 × 10¹⁰M cells | 4.98 × 10¹⁰M cells |
| of M cells | | | |
| Washing process | Horizontal | Horizontal | Countercurrent gradient |
| | centrifugal | centrifugal | centrifugal washing |
| | washing process | washing process | process |
| Specification | 5 × 10⁷ cells (1 mL/vial) | | |
| Batch yield | 939 vials | 952 vials | 945 vials |

Combining the above-mentioned production process, scale, batch output and other information of different batches, the results showed that both the horizontal centrifugal washing process and the countercurrent gradient centrifugal washing process could obtain good yield, and could ensure the operation time limit of each procedure, the batch yield was basically the same, and the process robustness was good.

### 1.7 Quality Evaluation

Three batches of M cell formulations were produced according to the above process. Samples were taken after 3 days of cryopreservation with vapor-phase liquid nitrogen to detect cell viability, density, MSC surface markers, biological activity of activating IDO-1, residual bovine serum albumin, sterility, mycoplasma and endotoxin.

### 1.7.1 Detection of Surface Markers

Based on the general definition requirements of ISCT for MSCs, the flow cytometry was used to detect cell surface marker molecules CD73, CD90, CD105, CD11b, CD19, CD34, CD45 and HLA-DR.

The detection results of three batches were shown in the following table:

| | Detection results of markers of three | | |
|---|---|---|---|
| | batches of cell formulation | | |
| Marker | (positive rate) | | |
| molecule | Batch 01 | Batch 02 | Batch 03 |
| CD73 | 99.79% | 99.17% | 99.23% |
| CD90 | 96.38% | 99.70% | 99.80% |
| CD105 | 100.00% | 99.31% | 99.40% |
| CD34 | 0.41% | 0.10% | 0.10% |
| CD45 | 0.10% | 0.04% | 0.10% |
| CD11b | 0.05% | 0.04% | 0.09% |
| CD19 | 0.09% | 0.07% | 0.05% |
| HLA-DR | 0.00% | 0.01% | 0.02% |

The results showed that the CD73, CD90 and CD105 of the three batches of M cells were all ≥95%, showing positive expression, and the CD11b, CD19, CD34, CD45 and HLA-DR were all ≤2%, meeting the general standards of ISCT for the phenotype definition of MSCs.

### 1.7.2 Detection of Chromosome Karyotype

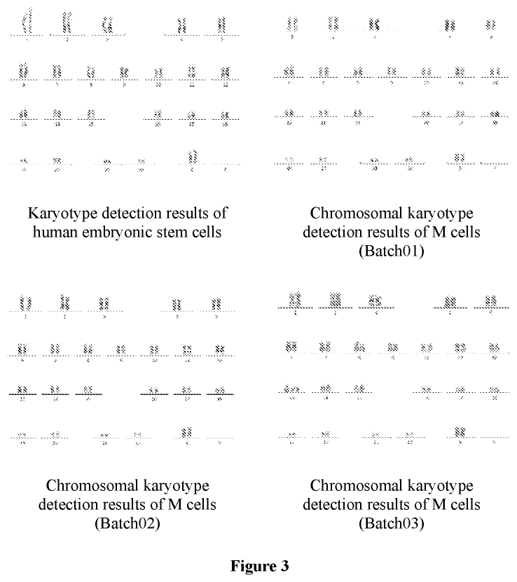

Chromosome karyotype analysis was performed on three consecutive batches of M cell formulations, and the results were shown in FIG. 3.

The results showed that all three batches of M cells were human cell lines, and they were from the same individual as the starting seed cells (human embryonic stem cells); all three batches of M cells had 46 normal chromosomes of human cells, and there was no chromosome deletion, translocation or aberration, indicating that the cell chromosomes were genetically stable during the entire differentiation and expansion culture process.

### 1.7.3 Isozyme Analysis

Isozyme detection was performed on three consecutive batches of M cell formulations, and the results were shown in the following table:

| | Batch 01 | Batch 02 | Batch 03 |
|---|---|---|---|
| Isozyme | Human cells, no cross | Human cells, no cross | Human cells, no cross |
| | contamination | contamination | contamination |

The results showed that all three batches of M cell formulations were human cells, and there was no cross contamination of cells from other species.

### 1.7.4 Cell Viability

Cell viability and viable cell number are the most basic and intuitive indicators to ensure that cells have biological efficacy, which are directly related to the therapeutic effects. In order to investigate the cell viability of the present product, the classic trypan blue exclusion method was used to determine the viability of three batches of M cell formulations. The results were shown in the following table:

Detection results of cell viability

| | Batch 01 | Batch 02 | Batch 03 |
|---|---|---|---|
| Cell viability (%) | 95.55% | 96.10% | 97.34% |

The results showed that the viability of the three batches of M cells was basically the same, and far higher than the general requirements for cryopreserved cell formulations in the FDA guidelines (cell viability should be ≥70%), that was, they had good cell viability.

### 1.7.5 Tri-Directional Differentiation Ability

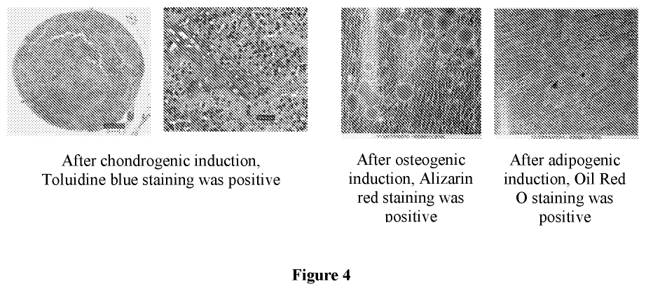

The differentiation ability of the three batches of M cells into adipocytes, osteoblasts and chondrocytes was detected. The detection results were shown in the following table, and a schematic diagram of the results off one batch was shown in FIG. 4.

| | Detection results of tridirectional differentiation ability | | |
|---|---|---|---|
| Item | Batch 01 | Batch 02 | Batch 03 |
| Adipogenic | After 21 days of | After 21 days of | After 21 days of |
| differentiation | adipogenic induction, | adipogenic induction, | adipogenic induction, |
| | Oil Red O staining was | Oil Red O staining was | Oil Red O staining was |
| | positive | positive | positive |
| Osteogenic | After 21 days of | After 21 days of | After 21 days of |
| differentiation | osteogenic induction, | osteogenic induction, | osteogenic induction, |
| | Alizarin red staining was | Alizarin red staining was | Alizarin red staining was |
| | positive | positive | positive |
| Chondrogenic | After 19 days of | After 19 days of | After 19 days of |
| differentiation | chondrogenic induction, | chondrogenic induction, | chondrogenic induction, |
| | Toluidine blue staining | Toluidine blue staining | Toluidine blue staining |
| | was positive | was positive | was positive |

The results showed that all the three batches of M cells had the ability to differentiate into adipogenic cells, osteogenic cells and chondrogenic cells, and the consistency between batches was good.

### 1.7.6 Immunomodulatory Ability

1.7.6.1 Level of L-Kynurenine from IDO-1 Activated by Inflammatory Factors

IDO1 (indoleamine 2,3-dioxygenase 1) is one of the important active molecules that mediate immune regulation function, and can promote the conversion of tryptophan to kynurenine, which not only consumes tryptophan in the cell environment, but also leads to the present production of kynurenine downstream metabolites, causing T cells to stagnate in the Gi phase, thereby inhibiting the proliferation of T lymphocytes and NK cells, and playing an important role in inhibiting inflammation.

Based on the fact that IDO1 can decompose tryptophan to generate L-kynurenine, and L-kynurenine can react with p-diaminobenzaldehyde under acidic conditions to generate p-diaminobenzaldehyde urea, which is a lemon-colored compound, the L-kynurenine content can be determined by chemical absorbance method. The detection method for activity of IDO1 secreted by the established M cells under IFN-γ stimulation was used to detect the IDO1 activity of the three batches of M cells. The detection results were shown in the following table:

Expression level of L-kynurenine

| | Batch 01 | Batch 02 | Batch 03 |
|---|---|---|---|
| IDO1 activity | 183 μmol/L | 171 μmol/L | 211 μmol/L |

The results showed that all the three batches of M cells could express immunomodulatory factor IDO1 with biological activity under the stimulation of inflammatory factor IFN-γ.

### 1.7.6.2 Immunoregulation of Lymphocytes and Subsets Thereof

MSCs can inhibit the proliferation and activation of pro-inflammatory T lymphocytes such as total lymphocytes, Th1 and Th17 subsets, and promote the proliferation of regulatory T cells (Treg) by directly interacting with different types of immune cells.

The regulatory function on various lymphocytes was detected by co-culturing the present product with total lymphocytes and lymphocyte subsets in combination with flow cytometry. The results were shown in FIG. 4.

The results showed that:
1) The M cells could inhibit the proliferation of activated total lymphocytes, and there was a dose-dependent effect;
2) The M cells could inhibit the proliferation of T cell subsets (Th1 and Th17), and there was a dose-dependent effect;
3) The M cells could promote the proliferation of regulatory T cells.

### 1.7.7 Detection of Tumorigenicity

### 1.7.7.1 Soft Agar Colony Formation Assay

In soft agar colony formation assay, the M cells were cultured continuously for 21 days to examine their cloning in the soft agar culture system to examine whether they had the possibility to exhibit tumorigenicity. The detection results of the three batches of M cells were shown in the following table:

| Item | Batch 01 | Batch 02 | Batch 03 |
|---|---|---|---|
| Detection of soft agar clone | No colony | No colony | No colony |
| formation | formation | formation | formation |

The results showed that the three batches of M cells did not have the ability to form soft agar clones, that was, they were not tumorigenic.

### 1.7.7.2 Telomerase Activity Method

Telomerase activity can reflect the self-renewal ability of cells to a certain extent, and differentiated cells usually maintain a low level. M cells are functional cells derived from the directed differentiation of human embryonic stem cells, and their telomerase activity is detected by the reverse transcription Telomeric Repeat Amplification Protocol (TRAP). By using the technology of fluorescence detection of telomerase activity based on TRAP principle, the total contents of samples and control cells were extracted for RT-qPCR quantitative detection, and the initial template amount of qPCR produced per unit of protein was calculated, and compared with A549 cells (human lung cancer alveolar basement epithelial cells), the relative telomerase activity of cells was calculated.

$$Telomerase activity of test sample relative to A 549 cells = \frac{1}{\begin{matrix}initial template amount produced per unit of A 549 protein / \\ initial template amount produced per unit of test sample protein\end{matrix}} \times 100 \%$$

The detection results of telomerase activity of the three batches of M cells were shown in the following table:

The results showed that the telomerase activity of the three batches of M cells was much lower than that of A549 cells, and the relative telomerase activity was about 10% of that of A549 cells, which were basically consistent.

### 1.7.7.3 In Vivo Tumorigenicity Test Method

Referring to Appendix 2 “Tumorigenicity Test Method” of Part III “Manufacture and Quality Control of Animal Cell Matrix for Production and Inspection of Biological Products” of “Chinese Pharmacopoeia”, 2020 edition, the three batches of M cells were tested for in vivo tumorigenicity. The cell suspensions were inoculated into 10 nude mice (10⁷ live cells/mouse), the mice were raised and the tumorigenicity was observed. The results were shown in the following table:

| | | Tumorigenicity test - in vivo inoculation method | | |
|---|---|---|---|---|
| | | Number of nude mice | Observation time | |
| Sample name | | inoculated (mice) | (days) | Result |
| Negative control group (2BS cells) | | 10 | 124 | No tumorigenicity |
| Positive control group (ascites) | | 10 | 21 | Had tumorigenicity |
| Test sample | Batch 01 | 10 | 124 | No tumorigenicity |
| | Batch 02 | 10 | 124 | No tumorigenicity |
| | Batch 03 | 10 | 124 | No tumorigenicity |

The results showed that the three batches of M cells were all negative in the in vivo tumorigenicity test, indicating that they were non-tumorigenic.

Combining the tumorigenicity test results of the above in vivo and in vitro methods (telomerase activity, soft agar cloning test), the three batches of M cells were non-tumorigenic.

### 1.7.8 Detection of Residual Non-Target Cells

In the present production process of the present product, human embryonic stem cells were placed in a specified culture system to simulate the human embryonic development process and directionally differentiate them into MSC through embryoid bodies. Non-target cells could include human embryonic stem cell residues, ectoderm cells, endoderm cells, and other non-target cells in mesoderm. The RT-qPCR method was used to evaluate the possibility of residual human embryonic stem cells; the RT-PCR method was used to detect the residual situation of other non-target cells, among which the characteristic genes PAX6 and OTX2 of neuroectodermal cells were selected as representatives of ectoderm cells; the characteristic genes FOXA2 and SOX17 in the differentiation process of hepatic progenitor cells were selected as representatives of endoderm cells; the characteristic genes cTNT and ISL1 of myocardial progenitor cells, and the characteristic genes CD45 and CD38 of hematopoietic stem cells were selected as representatives of other mesoderm-derived non-target cells. The results were detailed in the table below:

| | | | Detection result of non-target cells | | |
|---|---|---|---|---|---|
| Detection item and method | | | Batch 01 | Batch 02 | Batch 03 |
| Human embryonic | Expression level (2-ΔΔCp) | OCT4 | 1.09 | 1.85 | 0.90 |
| stem cell residue | relative to human foreskin | NANOG | 1.88 | 1.88 | 1.24 |
| (RT-qPCR method) | fibroblast (HFF) | | | | |
| Inspection of | Ectodermal cells | PAX6 | Not detected | Not detected | Not detected |
| unexpected | | OTX2 | Not detected | Not detected | Not detected |
| differentiation cells | Endoderm cells | FOXA2 | Not detected | Not detected | Not detected |
| (RT-PCR method) | | SOX17 | Not detected | Not detected | Not detected |
| | Mesoderm - Myocardial | cTNT | Not detected | Not detected | Not detected |
| | progenitor cells | ISL1 | Not detected | Not detected | Not detected |
| | Mesoderm - | CD38 | Not detected | Not detected | Not detected |
| | Hematopoietic stem cells | CD45 | Not detected | Not detected | Not detected |

The results showed that the human embryonic stem cell pluripotency genes OCT4 and NANOG were expressed in trace amounts in the three batches of M cell formulations, which were equivalent to the expression levels of differentiated and mature human adult cells (human foreskin fibroblasts), and thus there was no risk of residues. Combined with the soft agar cloning test and nude mouse in vivo inoculation test of the three batches of the finished products, the tumorigenicity results were all negative, which further indicated that the three batches of M cell formulations did not contain human embryonic stem cells. The representative genes of other directions of endoderm, ectoderm and mesoderm were not detected in the three batches of the finished products, and no non-target differentiated cells was observed.

### 1.7.9 Detection of Bovine Serum Albumin

Because the components of the M cell culture medium contained bovine serum albumin, which was a heterologous component, it was required to be removed from the present product. Therefore, in the formulation research of the present product, the residual bovine serum albumin was removed through the washing process. According to 3411 of Part IV of the Chinese Pharmacopoeia, 2020 edition, the ELISA method was used to detect the amount of BSA residue in the three consecutive batches of M cell formulations. The results were shown in the following table:

| | Detection result | | |
|---|---|---|---|
| Item | Batch 01 | Batch 02 | Batch 03 |
| Amount of BSA residue | Not detected | Not detected | Not detected |

The results showed that the three batches of M cell formulations had no residual bovine serum albumin after washing, which fully met the requirement of no more than 50 ng/dose in the Chinese Pharmacopoeia, and the batch-to-batch consistency was good.

### 1.7.10 Detection of Sterility

According to the sterility detection method in 1101 of Part 4 of the Chinese Pharmacopoeia, 2020 edition, the three batches of M cell formulations were detected. The results showed that all the three batches of M cell formulations were free of bacterial growth, which met the requirements.

### 1.7.11 Detection of Endotoxin

According to the endotoxin gel limit method in 1143 of Part 4 of the Chinese Pharmacopoeia, 2020 edition, the three batches of M cell formulations were detected. The endotoxin detection results of the three batches of M cell formulations were all lower than 15 EU/ml, which met the requirements.

### 1.7.12 Detection of Mycoplasma

According to the culture method and the indicator cell culture method in 3301 of Part 4 of the Chinese Pharmacopoeia, 2020 edition, the three batches of M cell formulations were detected. The mycoplasma detection results of the three batches of M cell formulations were all negative, which met the requirements.

### 1.7.13 Detection of pH Value

According to the pH value detection method in 0631 of Part 4 of the Chinese Pharmacopoeia, 2020 edition, the three batches of M cell formulations were detected. The results were shown in the following table, which were in line with the expectations.

| | Batch 01 | Batch 02 | Batch 03 |
|---|---|---|---|
| pH value | 7.3 | 7.3 | 7.4 |

### 1.7.14 Detection of Osmotic Pressure

According to the “Freezing Point Depression Method” in 0632 of Part III of the Chinese Pharmacopoeia, 2020 edition, the osmotic pressure molar concentrations of the three batches of M cell formulations were determined, and the results were shown in the following table:

| | Batch 01 | Batch 02 | Batch 03 |
|---|---|---|---|
| Osmotic pressure molar | 1356 | 1408 | 1387 |
| concentration (mOsmol/kg) | | | |

The results showed that the osmotic pressure molar concentrations of the three batches of M cell formulations were basically consistent, which were in line with the expectations.

### 1.8 Summary

Three batches of M cell formulations were continuously produced according to the operation parameters of the above process steps 1.1 to 1.5, and the yields of the three batches were nearly 1,000 vials. After inspection, the three batches of M cell formulations produced in large quantities had the basic biological characteristics of MSCs, differentiation ability, and immune regulation ability, still maintained high activity and viability after freezing, showed no tumorigenicity, and were free of process-related impurities (bovine serum albumin) after washing. The results showed that the formulation process of the present invention was robust and advantageous in controllable quality.

### Example 2: Research on Formulation

According to the formulation preparation process of the present product, 38 formulations were investigated in this research, and the stability of M cells in different formulations after room temperature placement and cryopreservation was evaluated.

The representative formulations and research results were listed as follows:

### 2.1 Formulation Composition

Each of the different formulations consisted of two parts: M cells and formulation solution, in which the M cells were prepared according to the present production process; and the formulation solution were functionally divided into three categories: crystalloid solution, non-permeable protective agent and permeable protective agent.

Among which, there were two main types of crystalloid solutions, Base 01 was an ionic salt solution with acid-base regulating ability, and Base 02 did not contain a pH regulator. Their specific components and contents thereof were shown in the following table:

| | Crystalloid solution | | |
|---|---|---|---|
| Component | | Base 01 | Base 02 |

| HEPES | 50 | mM | — | |
|---|---|---|---|---|
| Potassium chloride | 65 | mM | 5 | mM |
| Sodium chloride | 190 | mM | 90 | mM |
| Calcium chloride | 0.1 | mM | — | |
| Magnesium chloride | 10 | mM | 3 | mM |
| Potassium dihydrogen | 20 | mM | — | |
| phosphate | | | | |

| | | | |
|---|---|---|---|
| Sodium bicarbonate | 10 | mM | — |

| | | | |
|---|---|---|---|
| Sodium lactate | — | 27 | mM |
| Sodium gluconate | — | 23 | mM |

Formulation preparation: Base01 and Base02 were used as base solutions, and different permeable and non-permeable protective agents were added to prepare different formulation solutions, and M cells were diluted to a density of about 5×10⁷ cells/ml.

The components of each formulation were detailed in the table below:

| | | Formulation | | | | | | | | | | | | | | | | |
|---|---|---|---|---|---|---|---|---|---|---|---|---|---|---|---|---|---|---|
| Function | Component | F01 | F02 | F03 | F04 | F06 | F20 | | F23 | | F26 | F27 | F28 | F29 | F35 | | F36 | |
| Crystalloid | Base 01 | — | — | — | — | — | 50% | | 50% | | — | — | — | — | 50% | | 50% | |
| solution | Base 02 | 60% | 65% | 68% | 70% | 72% | — | | — | | 65% | 75% | 85% | 90% | — | | — | |
| Non-permeable | Glutathione | — | — | — | — | — | 3 | mM | 3 | mM | — | — | — | — | 3 | mM | 3 | mM |
| protective | Dextran-40 | — | — | — | — | — | 6% | | 6% | | — | — | — | — | 6% | | 6% | |
| agent | Adenosine | — | — | — | — | — | 2 | mM | 2 | mM | — | — | — | — | 2 | mM | 2 | mM |
| | Sucrose | — | — | — | — | — | 20 | mM | 20 | mM | — | — | — | — | 20 | mM | 20 | mM |
| | Mannitol | — | — | — | — | — | 20 | mM | 20 | mM | — | — | — | — | 20 | mM | 20 | mM |
| | Dextrose | — | — | — | — | — | 5 | mM | 5 | mM | — | — | — | — | 5 | mM | 5 | mM |
| | 20% human | 25% | 25% | 25% | 25% | 25% | — | | — | | — | — | — | — | — | | — | |
| | albumin | | | | | | | | | | | | | | | | | |
| | Compound | — | — | — | — | — | — | | — | | 30% | 20% | 10% | 5% | 5% | | 2% | |
| | amino acids | | | | | | | | | | | | | | | | | |
| Permeable | DMSO | 15% | 10% | 8% | 5% | 3% | 10% | | 5% | | 5% | 5% | 5% | 5% | 5% | | 5% | |
| protective | | | | | | | | | | | | | | | | | | |
| agent | | | | | | | | | | | | | | | | | | |
| Active | M cells | 5 × 10⁷ | | | | | | | | | | | | | | | | |
| ingredient | | | | | | | | | | | | | | | | | | |

### 2.2 Research Results

### 2.2.1 Stability of Placement Under Room Temperature Conditions

The changes in cell viability of the following different formulations placed at room temperature for different periods of time were investigated, and the results were shown in the following table:

| Formu- | Cell viability (%) | | | | | |
|---|---|---|---|---|---|---|
| lation No. | 0 min | 30 min | 60 min | 2 h | 3 h | 4 h |
| F01 | 93.02 | 95.99 | 97.06 | 93.82 | 92.72 | 93.59 |
| F02 | 96.62 | 96.64 | 98.04 | 97.27 | 95.24 | 97.59 |
| F03 | 98.40 | 98.32 | 98.35 | 98.70 | 97.07 | 96.80 |
| F04 | 98.22 | 99.15 | 98.56 | 97.56 | 97.25 | 97.34 |
| F06 | 96.09 | — | — | 96.03 | — | 98.13 |
| F20 | 94.03 | — | — | 96.71 | — | 95.75 |
| F23 | 96.67 | — | — | 95.89 | — | 94.38 |
| F26 | 93.37 | — | — | 94.22 | — | 92.85 |
| F27 | 95.87 | — | — | 94.80 | — | 93.62 |
| F28 | 95.01 | — | — | 94.04 | — | 95.53 |
| F29 | 94.52 | — | — | 93.82 | — | 92.76 |
| F35 | 95.57 | — | — | 96.05 | 95.68 | 96.02 |
| F36 | 97.24 | — | — | 96.75 | 97.02 | 95.98 |

The results showed that: for different formulation solutions, the cell viability did not change much over time, but the cells could have preferences for different formulation systems. The M cells were relatively more stable for the crystalloid solution (Base01) with buffering capacity.

2.2.2 Effect of Cryopreservation on Cell Viability after Placement

Considering the process from formulation preparation to filling requires nearly 3 hours during batch production of the present product, the formulation needed to be relatively stable for at least 3 hours. Therefore, a placement stability study was conducted on different formulations to observe the change in cell viability 3 hours after formulating preparation and after cell cryopreservation.

Cell viability (%)

| Formulation | Formulation placed | After placed for 3 hours |
|---|---|---|
| No. | for 3 hours | and cryopreservation |
| F01 | 93.59 | 84.14 |
| F02 | 97.59 | 87.04 |
| F03 | 96.80 | 90.11 |
| F04 | 97.34 | 93.55 |
| F06 | 98.13 | 93.79 |
| F20 | 95.75 | 91.40 |
| F23 | 94.38 | 94.72 |
| F26 | 92.85 | 91.54 |
| F27 | 94.62 | 91.04 |
| F28 | 95.53 | 88.42 |
| F29 | 92.76 | 90.17 |
| F35 | 96.02 | 93.93 |
| F36 | 95.98 | 92.89 |

The results showed that different formulations showed different sensitivities to cryopreservation and cooling after being placed at room temperature, which were specifically described as follows:
The higher the DMSO concentration, the more sensitive the M cell viability change, so the DMSO concentration should not be higher than 10%;
The crystalloid solution with buffering capacity, i.e., Base01 as contained in the formulations F20, F23, F35, and F36, brought higher stability than that of the non-buffering Base02 group;
The different combinations of non-permeable protective agents all had good protective ability for M cells, and there was no significant difference between the groups;
The formulations F23, F35, and F36 are free of human-derived components, and the viability before and after cryopreservation was relatively higher and more stable, so they were the preferred formulation components of the present product.

### Example 3: Research on Resuspension-Washing Solution

In order to fully remove the cell culture medium and digestion solution components, the resuspension-washing process was studied, and the resuspension solution and washing solution used should minimize the risk of introducing new impurities, such as human- or animal-derived components, new recombinant proteins and other bioactive components, and the solutions should minimize the damage to M cells. Therefore, the selection of resuspension solution and washing solution was studied based on the screening results of formulation F23, which specifically involved the following 9 components:

### 3.1 Screening of Resuspension-Washing Solution Based on Formulation F23

| | | R4 | R8 | R9 | R11 | R13 | R14 | R16 | R17 | R18 | R19 | R20 |
|---|---|---|---|---|---|---|---|---|---|---|---|---|
| Balance | HEPES (mM) | — | — | — | — | 5 | 10 | 5 | — | 5 | 10 | 30 |
| buffer | Potassium | 4.5 | 5 | — | 4 | 11 | 17 | 10 | 4 | 10 | 16.2 | 28.8 |
| | chloride | | | | | | | | | | | |
| | (mM) | | | | | | | | | | | |
| | Sodium | 81 | 90 | 102 | 102 | 110 | 120 | 109 | 102 | 109 | 120 | 147 |
| | chloride | | | | | | | | | | | |
| | (mM) | | | | | | | | | | | |
| | Calcium | — | — | — | 3 | — | — | 2.7 | 3 | 2.7 | 2.4 | 1.8 |
| | chloride | | | | | | | | | | | |
| | (mM) | | | | | | | | | | | |
| | Magnesium | 2.7 | 3 | — | — | 3.7 | 4.4 | 1 | — | 1 | 2 | 4 |
| | chloride | | | | | | | | | | | |
| | (mM) | | | | | | | | | | | |
| | Potassium | — | — | — | — | 2 | 4 | 2 | — | 2 | 4 | 8 |
| | dihydrogen | | | | | | | | | | | |
| | phosphate | | | | | | | | | | | |
| | (mM) | | | | | | | | | | | |
| | Sodium | — | — | — | — | 1 | 2 | 1 | — | 1 | 2 | 4 |
| | bicarbonate | | | | | | | | | | | |
| | (mM) | | | | | | | | | | | |
| | Sodium | 24 | 27 | | 27 | 25.2 | 22.4 | 27 | 27 | 25.2 | 22.4 | 19.6 |
| | lactate | | | | | | | | | | | |
| | (mM) | | | | | | | | | | | |
| | Sodium | 21 | 23 | — | — | 21 | 19 | | — | — | — | — |
| | gluconate | | | | | | | | | | | |
| | (mM) | | | | | | | | | | | |
| Protective | Glutathione (mM) | — | — | — | — | 0.3 | 0.6 | 0.3 | — | 0.3 | 0.6 | 1.2 |
| agent | Dextran-40 (%) | — | — | — | — | 0.06 | 0.12 | 0.06 | — | 0.06 | 0.12 | 0.18 |
| | Adenosine (mM) | — | — | — | — | 0.2 | 0.4 | 0.2 | — | 0.2 | 0.4 | 0.8 |
| | Sucrose (mM) | — | — | — | — | 2 | 4 | 2 | — | 2 | 4 | 8 |
| | Mannitol (mM) | — | — | — | — | 2 | 4 | 2 | — | 2 | 4 | 8 |
| | Dextrose (mM) | — | — | — | — | 0.5 | 1 | 1 | — | 0.5 | 1 | 2 |
| | OsrHSA(%) | — | — | — | — | — | — | — | 10 | — | — | — |
| | Compound amino | 10 | — | — | — | — | — | — | — | — | — | — |
| | acids (%) | | | | | | | | | | | |

In order to ensure that the operation time was long during the mass production, the resuspension and washing solutions were studied in combination with the stability at room temperature. After resuspension, the cells were placed for 1 to 4 hours, resuspended and washed 1 to 3 times, the total number of cells was determined, and the formulation solution was added to prepare an M cell formulation containing about 5×10⁷ cells/ml, which was packaged and cryopreserved, and the cell viability was detected after 3 days of vapor-phase liquid nitrogen equilibrium.

The results of cell viability detection were shown in the following table:

| Resuspension | | | Washing | |
|---|---|---|---|---|
| Resuspension- | | | Resuspension- | Resuscitation |
| washing | Placement | Cell | washing | Cell |
| solution No. | time | viability (%) | solution No. | viability (%) |
| R4 | 0 h | 95.92 | R4 | 87.25 |
| | 2 h | 96.62 | | 86.21 |
| | 4 h | 95.64 | | 83.27 |
| R8 | 0 h | 93.35 | R8 | 91.53 |
| | 2 h | 97.31 | | 88.20 |
| | 4 h | 97.26 | | 83.41 |
| R9 | 0 h | 97.40 | R9 | 86.26 |
| | 2 h | 97.90 | | 86.17 |
| | 4 h | 97.71 | | 82.01 |
| R11 | 0 h | 97.27 | R8 | 94.91 |
| | 3 h | 97.16 | | 89.15 |
| R13 | 0 h | 97.97 | R8 | 90.02 |
| | 2 h | 98.26 | | 89.28 |
| | 4 h | 98.01 | | 88.20 |
| R14 | 0 h | 98.02 | R8 | 91.36 |
| | 2 h | 97.52 | | 91.38 |
| | 4 h | 97.55 | | 85.29 |
| R14 | 0 h | 97.98 | R14 | 89.47 |
| | 2 h | 97.85 | | 86.27 |
| | 4 h | 96.94 | | 85.56 |
| R16 | 0 h | 96.56 | R16 | 95.25 |
| | 2 h | 97.81 | | 94.97 |
| | 4 h | 97.75 | | 93.64 |
| R16 | 0 h | 97.09 | R17 | 92.37 |
| | 2 h | 98.22 | | 91.66 |
| | 4 h | 97.81 | | 90.11 |
| R17 | 0 h | 98.57 | R11 | 90.80 |
| | 2 h | 98.85 | | 89.38 |
| | 4 h | 98.79 | | 85.29 |
| R17 | 0 h | 97.72 | R17 | 88.92 |
| | 2 h | 98.75 | | 85.94 |
| | 4 h | 98.58 | | 82.19 |
| R18 | 0 h | 96.71 | R18 | 96.18 |
| | 2 h | 97.60 | | 95.27 |
| | 4 h | 96.10 | | 94.24 |
| R18 | 0 h | 97.20 | R19 | 93.71 |
| | 2 h | 98.03 | | 94.38 |
| | 4 h | 97.46 | | 94.47 |
| R19 | 0 h | 97.82 | R19 | 94.39 |
| | 2 h | 97.53 | | 94.24 |
| | 4 h | 97.28 | | 93.37 |
| R20 | 0 h | 98.20 | R20 | 95.72 |
| | 2 h | 97.89 | | 94.26 |
| | 4 h | 97.82 | | 93.48 |

The results showed that:

When R16, R18, R19 and R20 in the groups were used as resuspension and washing solutions, it was ensured that the cells could be stably placed for at least 4 hours, and had a cell viability of not less than 93%, and the others in the groups had weaker protective effects, but could still maintain a cell viability of not less than 80%, which met the FDA's requirement that the cell viability should be not less than 70% for cell products.

It could be seen that the M cells had a tendency to tolerate different resuspension solutions and washing solutions. In general, compositions incorporating colloidal stabilizers (e.g., sucrose, mannitol, dextrose), ionic/pH regulators (e.g., disodium hydrogen phosphate, sodium bicarbonate, sodium lactate), and metabolic stabilizers (e.g., glutathione, adenosine) demonstrate superior cytoprotective efficacy for the M cells, effectively mitigating cellular damage during extended holding periods.

### Example 4: Research on Washing Process

The culture medium introduced into the present product during the cell culture process contained bovine serum albumin (BSA), which was a heterologous component and needed to be removed from the final product. In addition, when harvesting the M cells, the digestive enzyme CTS TrypLE was used, while this component could degrade nucleic acids and posed a human safety risk, which also needed to be removed from the final product. Therefore, a study on washing process was carried out to determine the washing conditions.

Two washing processes were studied, which were based on horizontal centrifugation process and countercurrent gradient centrifugation process, respectively, and detailed are as follows:

### 4.1 Study on Horizontal Centrifugation Process

The harvested M cells were taken and resuspended in three resuspension-washing solutions R18\R19\R20, respectively, and washed 3 times, in which the solution of washing volume was added at 200 mL/cell factory each time. The residual amounts of BSA and TrypLE in the cell formulations and the cell viability after different washing times were detected, and the results were shown in the following table:

| | | Results | | | |
|---|---|---|---|---|---|
| | | Cell | Biological | TrypLE | BSA |
| | | viability | activity | residue | residue |
| Resuspension-washing solution | | (%) | (μmol/L) | (ng/mL) | (ng/mL) |
| R18 | Cell resuspension | — | — | 529.29 | 236.23 |
| | Cryopreservation | 94.24 | 301 | 4.106 | 6.32 |
| | formulation after | | | | |
| | washing once | | | | |
| | Cryopreservation | 93.94 | 315 | 3.556 | 5.19 |
| | formulation after | | | | |
| | washing twice | | | | |
| | Cryopreservation | 94.71 | 285 | 2.561 | 4.28 |
| | formulation after | | | | |
| | washing 3 times | | | | |
| R19 | Cell resuspension | — | — | 580.44 | 269.45 |
| | Cryopreservation | 94.62 | 283 | 3.731 | 7.397 |
| | formulation after | | | | |
| | washing once | | | | |
| | Cryopreservation | 92.82 | 304 | 3.322 | 4.834 |
| | formulation after | | | | |
| | washing twice | | | | |
| | Cryopreservation | 95.51 | 319 | 3.368 | 5.954 |
| | formulation after | | | | |
| | washing 3 times | | | | |
| R20 | Cell resuspension | — | — | 583.77 | 298.12 |
| | Cryopreservation | 93.95 | 320 | 4.821 | 6.823 |
| | formulation after | | | | |
| | washing once | | | | |
| | Cryopreservation | 92.87 | 309 | 3.981 | 4.015 |
| | formulation after | | | | |
| | washing twice | | | | |
| | Cryopreservation | 94.56 | 313 | 3.241 | 4.718 |
| | formulation after | | | | |
| | washing 3 times | | | | |

The results showed that:

Three resuspension-washing solutions with different components were selected to wash M cells 1 to 3 times. After washing, the residual amounts of TrypLE and BSA in the formulations were significantly reduced, but with the increase in the number of washes, the residual amounts did not decrease significantly, while the cell viability and activity did not decrease, and the residual amount of BSA was far below the standard of ≤50 ng/mL in the Chinese Pharmacopoeia. Therefore, washing 1 to 3 times could be considered to be the washing process, and the washing volume was not less than 200 mb/cell factory.

### 4.2 Study on Washing Process by Countercurrent Gradient Centrifugation Method

Referring to the process steps, the washing process adopted the automatic Rotea countercurrent centrifugation system, which was a closed operation system and could effectively reduce the risk of contamination caused by manual operation.

The parameters for setting the countercurrent centrifugation system were as follows:
Washing solution volume: washing was tried under four conditions of 80 mL to 200 mL for each cell factory;
Centrifugal force and flow rate for loading sample: at centrifugal force of 2800×g, the flow rate for loading sample was 45 mL/min;
Centrifugal force and flow rate for washing: at centrifugal force of 2500×g, 45 mL/min.

After the resuspension and washing by the countercurrent centrifugation system, the total amount of MSCs, cell viability, residual cell amount in waste liquid, and cell viability after cryopreservation and resuscitation were detected, and the process-related impurities before and after washing were detected. The test results were as follows:

| | | Washing volume | | | |
|---|---|---|---|---|---|
| | | 80 | 120 | 150 | 200 |
| Detection | | mL | mL | mL | mL |
| Total | After resuspension | 4.57 | 4.83 | 4.92 | 4.58 |
| cell amount | After washing | 3.68 | 3.67 | 3.50 | 3.11 |
| (×10⁸) | Residual cell | 0.06 | 0.05 | 0.05 | 0.04 |
| | amount in | | | | |
| | waste liquid | | | | |
| Recovery rate (%) | | 80.53 | 78.98 | 75.13 | 78.90 |
| Cell | After resuspension | 97.51 | 97.46 | 96.82 | 96.37 |
| viability | After washing | 95.77 | 95.61 | 96.02 | 95.27 |
| (%) | Formulation | 94.26 | 93.14 | 95.02 | 93.28 |
| Residual | After resuspension | 723.206 | 682.836 | 728.920 | 829.027 |
| amount of | After washing | 21.308 | 10.978 | 4.649 | 5.017 |
| TrypLE | Formulation | 9.810 | 7.201 | 7.592 | 8.027 |
| (ng/mL) | | | | | |

Under the above experimental conditions, the cell recovery rate was maintained at about 80%, and the cell viability after cryopreservation was maintained at more than 90%. The residual amount of TrypLE showed a decreasing trend with the increase of washing volume, especially after washing with 150 mL and 200 mL volume, and the finished product met expectations. Therefore, the countercurrent gradient washing process could effectively remove impurities and maintain a high cell viability, and could be applied to large-scale, automated batch production.

### Example 5: Study on Programmed Freezing Process

During the cryopreservation process of cells, a too fast cooling rate can easily lead to the formation of intracellular ice, causing cell damage; if the cooling rate is too slow, the cells will lose water severely, resulting in a “solution effect” and causing damage to the cells. In order to ensure that the cells still maintained a high viability after cryopreservation and resuscitation, the cooling program of the present product was studied. The cooling instrument used was 7451TF model of Thermofisher Company, and the following 12 cooling programs were studied:

| | | | Step3 | | Step4 | | Step5 | | Step6 | | | |
|---|---|---|---|---|---|---|---|---|---|---|---|---|
| | | | | until | | until | | until | | until | | |
| | Step1 | Step2 | Ramp | Chamber | Ramp | Chamber | Ramp | Chamber | Ramp | Chamber | Step7 | Step8 |
| C1 | Wait at | Wait at | 1° C./ | −40° C. | — | — | — | — | — | — | Ramp | Hold |
| | 4° C. | Chamber | min | | | | | | | | 10.0° C./ | −90.0° C. |
| C2 | | 4° C. | 0.5° C./ | −40° C. | — | — | — | — | — | — | min until | for 10.0 |
| | | until | min | | | | | | | | Chamber = | minutes |
| C3 | | sample | 2° C./ | −40° C. | — | — | — | — | — | — | −90.0° C. | |
| | | 5° C. | min | | | | | | | | | |
| C4 | | | 1° C./ | −13° C. | 25° C./ | −55° C. | 10° C./ | −20° C. | 1° C./ | −40° C. | | |
| | | | min | | min | | min | | min | | | |
| C5 | | | 1° C./ | −13° C. | 30° C./ | −55° C. | 10° C./ | −20° C. | 1° C./ | −40° C. | | |
| | | | min | | min | | min | | min | | | |
| C6 | | | 1° C./ | −13° C. | 20° C./ | −55° C. | 10° C./ | −20° C. | 1° C./ | −40° C. | | |
| | | | min | | min | | min | | min | | | |
| C7 | | | 1° C./ | −12° C. | 25° C./ | −55° C. | 10° C./ | −20° C. | 1° C./ | −40° C. | | |
| | | | min | | min | | min | | min | | | |
| C8 | | | 1° C./ | −8° C. | 20° C./ | −55° C. | 10° C./ | −20° C. | 1° C./ | −40° C. | | |
| | | | min | | min | | min | | min | | | |
| C9 | | | 0.8° C./ | −13° C. | 25° C./ | −55° C. | 10° C./ | −20° C. | 1° C./ | −40° C. | | |
| | | | min | | min | | min | | min | | | |
| C10 | | | 1.2° C./ | −13° C. | 25° C./ | −55° C. | 10° C./ | −20° C. | 1° C./ | −40° C. | | |
| | | | min | | min | | min | | min | | | |
| C11 | | | 1° C./ | −13° C. | 25° C./ | −55° C. | 12° C./ | −20° C. | 1° C./ | −40° C. | | |
| | | | min | | min | | min | | min | | | |
| C12 | | | 1° C./ | −13° C. | 25° C./ | −55° C. | 9° C./ | −20° C. | 1° C./ | −40° C. | | |
| | | | min | | min | | min | | min | | | |

After the programmed freezing, the cells were transferred into a vapor-phase liquid nitrogen tank for cryopreservation, and taken out after stabilization for 3 days. The cell viability before and after cryopreservation of M cell formulations and the cell biological activity after resuscitation were detected, and the detection results were as follows:

| | Cell viability | Cell viability | |
|---|---|---|---|
| Cooling | before | after | Biological |
| program | cryopreservation | resuscitation | activity |
| No. | (%) | (%) | (μmol/L) |
| C1 | 95.24 | 93.38 | 301.26 |
| C2 | 96.83 | 91.29 | 283.91 |
| C3 | 96.28 | 83.26 | 238.28 |
| C4 | 95.83 | 93.29 | 302.06 |
| C5 | 96.58 | 94.96 | 327.29 |
| C6 | 97.17 | 94.28 | 309.25 |
| C7 | 95.10 | 93.38 | 328.28 |
| C8 | 97.27 | 95.01 | 305.27 |
| C9 | 95.83 | 93.03 | 286.93 |
| C10 | 96.37 | 94.71 | 322.01 |
| C11 | 97.31 | 95.06 | 299.38 |
| C12 | 96.82 | 94.02 | 332.75 |

The results showed that, except for the C3 program, the above 12 cooling programs showed a difference of about 2% in cell viability among the groups before and after the programmed freezing, which could ensure that the cell viability was not less than 90%, and there was no significant difference in cell biological activity among the groups.

### Example 6: Study on Long-Term Stability

The shelf life of cell cryopreservation formulations needs to be determined based on long-term stability. According to the requirements of “Guidelines for Stability Tests of Biological Products” in 9402 of “Chinese Pharmacopoeia”, 2020 edition, and the characteristics of the present product, a study on long-term stability was carried out under liquid nitrogen conditions, and the information of the study was summarized in the following table:

| | | | |
|---|---|---|---|
| Sample to | | Conditions | Vapor-phase liquid |
| be studied | Batch 01 | of study | nitrogen, ≤−150° C. |

| Sampling | Month 0, Month 3, Month 6, Month 9, | |
|---|---|---|
| points | Month 12, Month 18, Month 24, | |
| | Month 30, Month 36 | |
| Investigation | Identification, cell | |
| items | viability, biological activity | |
| Acceptable | Identification | CD73 should be ≥95% |
| standard | | CD90 should be ≥95% |
| | | CD105 should be ≥95% |
| | | CD34 should be ≤2% |
| | | CD45 should be ≤2% |
| | Cell viability | should be ≥90% |
| | Live cell density | Should be 3 to 7 × 10⁷ cells/mL |
| | Biological activity | L-kynurenine should be ≥50 μmol/L |

The detection results of the long-term stability of Batch 01 were shown in the following table:

| | | Detection results of long-term stability of | | | | | | | |
|---|---|---|---|---|---|---|---|---|---|
| | | Batch 01 at different time points | | | | | | | |
| | | Acceptable | Month | Month | Month | Month | Month | Month | Month |
| Item | | standard | 0 | 3 | 6 | 9 | 12 | 18 | 24 |
| Cell viability | | should be ≥90% | 95.55% | 95.04% | 95.77% | 95.32% | 97.37% | 95.37% | 96.23% |
| Identi- | CD73 | should be ≥95% | 99.79% | 99.61% | 98.56% | 97.82% | 99.29% | 99.68% | 99.57% |
| fication | CD90 | should be ≥95% | 96.38% | 99.84% | 99.73% | 98.84% | 99.64% | 99.87% | 99.53% |
| | CD105 | should be ≥95% | 100.00% | 99.50% | 99.90% | 99.29% | 99.74% | 99.96% | 99.82% |
| | CD34 | should be ≤2% | 0.41% | 0.15% | 0.27% | 0.24% | 0.29% | 0.39% | 0.018% |
| | CD45 | should be ≤2% | 0.10% | 0.10% | 0.11% | 0.07% | 0.03% | 0.04% | 0.06% |

| | | | | | | | | |
|---|---|---|---|---|---|---|---|---|
| Biological | L-kynurenine | 197 | 183 | 135 | 223 | 181 | 212 | 209 |
| activity | should be | | | | | | | |
| | ≥50 μmol/L | | | | | | | |

The above detection results showed that: during the long-term storage of the finished product for 24 months, the detection results of the product had no significant changes as compared with those of the 0th month, which met the requirements of the quality standards, indicating that the present product could be stably stored for at least 24 months under cryopreservation conditions.

### Example 7: Accelerated Study on Stability

The present product was a cryopreservation formulation, which could be temporarily out of the cold chain during transportation, transport or use. Therefore, the tolerance of the present product to the situation of being temporarily out of cold chain should be studied through an accelerated stability study.

The summary of the accelerated stability study was shown in the following table:

| | | | |
|---|---|---|---|
| Sample to | | Conditions | |
| be studied | Batch 01 | of study | −80 ± 10° C. |

| Sampling | Week 0, Week 1, Week 2, | |
|---|---|---|
| points | Week 4, Week 8 | |
| Inspection | Identification, cell | |
| items | viability, biological activity | |
| Acceptable | Identification | CD73 should be ≥95% |
| standard | | CD90 should be ≥95% |
| | | CD105 should be ≥95% |
| | | CD34 should be ≤2% |
| | | CD45 should be ≤2% |
| | Cell viability | should be ≥90% |
| | Live cell density | should be 3 to 7 × 10⁷ cells/mL |
| | Biological activity | L-kynurenine should be ≥50 μmol/L |

The results of the accelerated stability study were shown in the following table:

| | | | Accelerated stability test results of | | | | |
|---|---|---|---|---|---|---|---|
| | | Acceptable | Batch 01 at different time points | | | | |
| Item | | standard | Week 0 | Week 1 | Week 2 | Week 4 | Week 8 |
| Cell viability | | should be ≥90% | 95.04% | 91.67% | 92.57% | 93.52% | 94.75% |
| Identi- | CD73 | should be ≥95% | 99.61% | 98.51% | 98.45% | 98.77% | 98.04% |
| fication | CD90 | should be ≥95% | 99.84% | 99.54% | 99.70% | 99.78% | 99.76% |
| | CD105 | should be ≥95% | 99.50% | 99.89% | 99.79% | 99.83% | 99.90% |
| | CD34 | should be ≤2% | 0.15% | 0.08% | 0.38% | 0.41% | 0.26% |
| | CD45 | should be ≤2% | 0.10% | 0.07% | 0.28% | 0.17% | 0.26% |

| | | | | | | |
|---|---|---|---|---|---|---|
| Biological | L-kynurenine | 183 | 202 | 212 | 158 | 188 |
| activity | should be | | | | | |
| | ≥50 μmol/L | | | | | |

The above test results showed that the present product could be stably stored at −80±10° C. for at least 8 weeks, and all indicators had no significant changes, which met the quality standards.

### Example 8: Study on Stability in Clinical Blending

To ensure the quality of clinical use of the present product, the cells were diluted to 1×10⁶ to 5×10⁷ with physiological saline as solvent, and the stability during the placement at room temperature and 2 to 8° C. after the blending was investigated. The specific scheme of the study was summarized in the following table:

| | | | |
|---|---|---|---|
| | | | 5 ± 3° C. |
| Sample to | | Conditions | Room temperature |
| be studied | Batch 01 | of study | (20 ± 10° C.) |

| Blending | 5 × 10⁷ cells/mL | |
|---|---|---|
| concentrations | 2.5 × 10⁷ cells/mL | |
| | 1 × 10⁷ cells/mL | |
| | 5 × 10⁶ cells/mL | |
| | 1 × 10⁶ cells/mL | |
| Sampling points | 0 h, 2 h, 4 h, 6 h, 8 h | |
| Investigation item | Cell viability | |
| Acceptable standard | Cell viability | should be ≥90% |

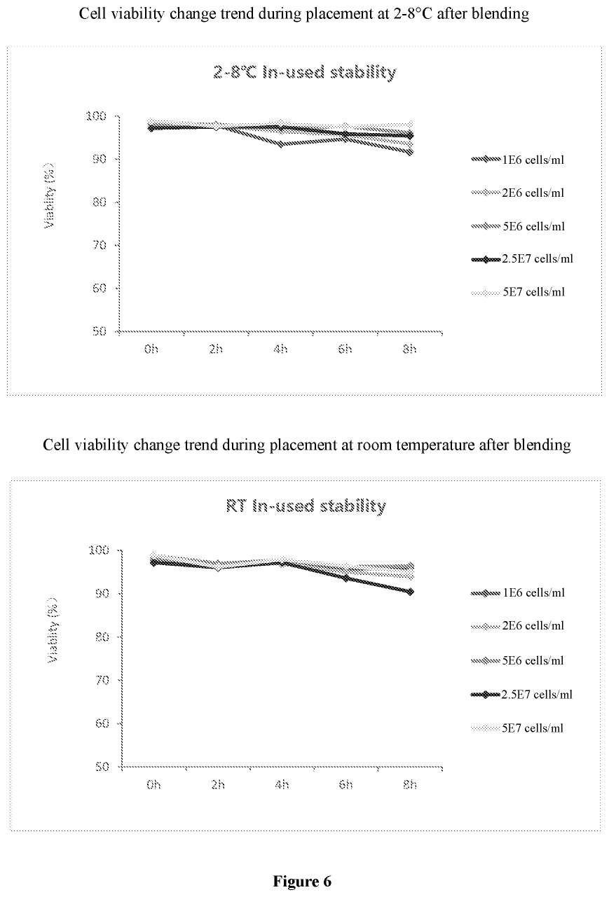

The detection results were shown in FIG. 6.

The above detection results showed that the cell viability at each dilution density after placement at 5±3° C. and room temperature (20±10° C.) for 8 h was in compliance with the quality standard. In order to prevent the cell viability from decreasing due to prolonged placement, it was recommended to infuse the cells as soon as possible after the clinical blending.

Although the present application has shown and described exemplary embodiments of the present invention, it will be understood by those skilled in the art that the above embodiments should not be construed as limiting the content of the present invention, and can be changed, replaced and modified without departing from the spirit, principle and scope of the content of the present invention.

1.-10.

11. An off-the-shelf stem cell formulation composition, which comprises MSCs (i.e., M cells) obtained by directed differentiation and expansion of human embryonic stem cells and an excipient, wherein the off-the-shelf stem cell formulation composition does not contain human-derived and animal-derived excipients, wherein the excipient comprises one or more of the following: permeable protective agent, non-permeable protective agent, crystal solution, and pH regulator, wherein the permeable protective agent is selected from DMSO, the non-permeable protective agent is one or more selected from the group consisting of adenosine, low-molecular-weight dextran, lactobionic acid, HEPES, reduced L-glutathione, glucose, sucrose, mannitol, compound amino acids, and plant-derived recombinant human serum albumin, and the crystal solution and pH regulator are one or more selected from the group consisting of sodium hydroxide, sodium gluconate, sodium acetate, potassium hydroxide, sodium chloride, calcium chloride, magnesium chloride, potassium chloride, potassium bicarbonate, potassium phosphate, calcium chloride, magnesium chloride.

12. The off-the-shelf stem cell formulation composition according to claim 11, wherein the stem cell formulation composition further comprises one or more of hydroxyethyl starch, SUL-109 (6-chromanol derivative), malate buffer, acetate buffer.

13. The off-the-shelf stem cell formulation composition according to claim 11, wherein the formulation composition has a viable cell density of about 5×10⁷ cells/ml, and wherein the formulation composition has a cell viability of >90%, wherein the formulation composition is substantially free of human embryonic stem cell residues, substantially free of bovine serum albumin residues and substantially free of non-target differentiated cells, and wherein the formulation composition is non-tumorigenic.

14. The off-the-shelf stem cell formulation composition according to claim 11, wherein the formulation composition can be stably stored for at least 24 months.

15. The off-the-shelf stem cell formulation composition according to claim 11, wherein the formulation composition can be stably placed at −80±10° C. for at least 8 weeks.

16. The off-the-shelf stem cell formulation composition according to claim 11, wherein the excipient comprises potassium chloride, sodium chloride, magnesium chloride, sodium lactate, sodium gluconate.

17. The off-the-shelf stem cell formulation composition according to claim 16, wherein the excipient further comprises glutathione, dextran-40, adenosine, sucrose, mannitol, dextrose, and DMSO.

18. A method for mass production of off-the-shelf stem cell formulations, which comprises:
a) harvesting MSCs, i.e., M cells, obtained by directed differentiation and expansion of human embryonic stem cells,
b) resuspending and washing the cells obtained in step a),
c) formulating the washed cells into a stem cell formulation at 2 to 8° C.,
d) filling the formulated stem cell formulation into a stem cell drug packaging material suitable for a smaller volume of cell formulation at 2 to 8° C.,
e) cooling the filled stem cell formulation to ≤−70° C.,
f) transporting and/or storing the obtained off-the-shelf stem cell formulation, wherein the off-the-shelf stem cell formulation does not contain human-derived and animal-derived excipients.

19. The method for mass production of off-the-shelf stem cell formulation according to claim 18, wherein step a) comprises washing the cultured cell with sodium lactate Ringer's solution, adding a digestion solution for digestion, and then stopping the digestion by adding a stop solution, wherein the digestion solution is CTS TrypLE, the stop solution comprises glutathione, dextran-40, adenosine, sucrose, mannitol, glucose, HEPES, potassium chloride, sodium chloride, calcium chloride, magnesium chloride, potassium dihydrogen phosphate, sodium bicarbonate, and sodium lactate.

20. The method for mass production of off-the-shelf stem cell formulation according to claim 18, wherein the resuspension in step b) comprises adding the M cell obtained in step a) to a resuspension solution for resuspension to obtain a cell suspension, and/or
wherein the washing in step b) comprises washing with a horizontal centrifuge or washing with a countercurrent gradient centrifuge.

21. The method for mass production of off-the-shelf stem cell formulation according to claim 18, wherein the resuspension solution and/or the washing solution comprises glutathione, dextran-40, adenosine, sucrose, mannitol, glucose, HEPES, potassium chloride, sodium chloride, calcium chloride, magnesium chloride, potassium dihydrogen phosphate, sodium bicarbonate, sodium lactate, dextrose.

22. The method for mass production of off-the-shelf stem cell formulation according to claim 21, wherein the resuspension solution and/or the washing solution comprises 1 to 40 mM HEPES, 1 to 30 mM potassium chloride, 80 to 130 mM sodium chloride, 1 to 6 mM calcium chloride, 1 to 5 mM magnesium chloride, 1 to 10 mM potassium dihydrogen phosphate, 1 to 8 mM sodium bicarbonate, 15 to 30 mM sodium lactate, 0.1 to 1.5 mM glutathione, 0.01% to 0.2% dextran-40, 0.1 to 1 mM adenosine, 1 to 10 mM sucrose, 1 to 10 mM mannitol, 0.1 to 4 mM dextrose.

23. The method for mass production of off-the-shelf stem cell formulation according to claim 18, wherein the washed cell is formulated into a stem cell formulation using a formulation solution, wherein the formulation solution comprises glutathione, dextran-40, adenosine, sucrose, mannitol, dextrose, HEPES, potassium chloride, sodium chloride, calcium chloride, magnesium chloride, potassium dihydrogen phosphate, sodium bicarbonate, and DMSO.

24. The method for mass production of off-the-shelf stem cell formulation according to claim 23, wherein the formulation solution comprises 20 to 30 mM HEPES, 30 to 35 mM potassium chloride, 90 to 100 mM sodium chloride, 0.01 to 0.1 mM calcium chloride, 1 to 10 mM magnesium chloride, 5 to 15 mM potassium dihydrogen phosphate, 1 to 10 mM sodium bicarbonate, 1 to 6 mM glutathione, 2% to 12% dextran-40, 1 to 4 mM adenosine, 15 to 25 mM sucrose, 15 to 25 mM mannitol, 1 to 10 mM dextrose, 1% to 15% DMSO.

25. The method for mass production of off-the-shelf stem cell formulation according to claim 23, wherein the formulation solution further comprises 1 to 10% compound amino acids.

26. The method for mass production of off-the-shelf stem cell formulation according to claim 18, wherein the programmed freezing comprises: waiting at 2° C. to 8° C.; cooling to −11° C. to −17° C. at 1° C. to 2° C./min; cooling to −45° C. to −65° C. at 20° C. to 30° C./min; heating to −15° C. to −25° C. at 8° C. to 16° C./min; cooling to −35° C. to −45° C. at 1° C. to 2° C./min; cooling to −80° C. to −90° C. at 8° C. to 12° C./min; maintaining at −80° C. to −90° C. for 10 min to 20 min.

27. The method for mass production of off-the-shelf stem cell formulation according to claim 18, wherein the programmed freezing comprises: waiting at 4° C.; cooling to −13.0° C. at 1° C./min; cooling to −55.0° C. at 25° C./min; heating to −20.0° C. at 12° C./min; cooling to −40.0° C. at 1.0° C./min; cooling to −90.0° C. at 10.0° C./min; maintaining at −90.0° C. for 10 min.

28. An off-the-shelf stem cell formulation produced by the method according to claim 18.

29. A method for treating an indication, comprising administering the off-the-shelf stem cell formulation composition according to claim 11 or the off-the-shelf stem cell formulation according to claim 18 by local injection or intravenous infusion, wherein the indication comprises but is not limited to: osteoarthropathy, reproductive system disease, heart disease, lung disease, skin disease, eye disease, nervous system disease, digestive system disease, kidney disease, liver disease, autoimmune disease, transplant rejection, metabolic disease.

30. The method of claim 29, wherein the osteoarthropathy is meniscus injury, osteoarthritis, or bone injury; the reproductive system disease is ovarian aging, ovarian insufficiency, endometrial injury, uterine trauma, intrauterine adhesion, or thin uterus; the heart disease is myocardial infarction; the lung disease is idiopathic pulmonary fibrosis, acute respiratory distress syndrome, pneumoconiosis, or pneumonia; the skin disease is psoriasis, skin injury, bedsore, pressure sore, or burn; the eye disease is corneal injury; the nervous system disease is spinal cord injury, cerebral palsy, stroke, Alzheimer's disease, or neuropathic pain; the digestive system disease is inflammatory bowel disease, colitis, Crohn's disease, or irritable bowel syndrome; the kidney disease is anti-glomerular basement membrane disease, diabetic nephropathy, lupus nephritis, or acute nephritis; the liver disease is liver injury, liver fibrosis, hepatitis, cirrhosis, or liver failure; the autoimmune disease is scleroderma, lupus erythematosus, or multiple sclerosis; the transplant rejection is graft-versus-host disease; and the metabolic disease is diabetes.
